(19) United States
(12) Patent Application Publication
(10) Pub. No.: US 2006/0122132 A1
(43) Pub. Date: Jun. 8, 2006

(54) Use of murine genomic regions identified to be involved in tumor developement for the development of anti-cancer drugs and diagnosis of cancer

(76) Inventors: Ivo Paul Touw, Le Rotterdam (NL); Hendrik Rudolf Delwel, Westmaas (NL); Bob Lowenberg, Jp Rotterdam (NL); Peter Jacobus Maria Valk, Rotterdam (NL)

Correspondence Address: THE WEBB LAW FIRM, P.C., 700 KOPPERS BUILDING, 436 SEVENTH AVENUE, PITTSBURGH, PA, 15219, US

(21) Appl. No.: 10/524,507

(22) PCT Filed: Aug. 14, 2003
(86) PCT No.: PCT/NL03/00583

§ 371 Date: Sep. 29, 2005

(30) Foreign Application Priority Data
Aug. 14, 2002 (EP) 02078358.5
Sep. 14, 2002 (US) 10252132

(51) Int. Cl. A61K 48/00; C12Q 1/68; C07H 21/04; C12P 21/06; A61K 39/395; C07K 14/82; C12N 5/06

(52) U.S. Cl. 514/44; 424/155.1; 435/69.1; 435/354; 435/320.1; 530/388.8; 530/350; 536/23.2; 435/6

(57) ABSTRACT

The present invention relates to murine genomic regions, identified by retroviral insertional tagging of mice as being involved in tumor development, in particular leukemia development, as well as human homologues thereof, and to the use of these genomic regions for the identification and development of anti-cancer drugs, such as small molecule inhibitors, antibodies, ribozymes, antisense molecules and RNA interference (RNAi) molecules, that are effective in reducing or eliminating the tumorigenic effects of genetic transformations in these genomic regions and/or eliminating the tumorigenic effects of expression products thereof. The invention further relates to these anti-cancer drugs and to their use as pharmaceutical reagents for the treatment of cancer, as well as to pharmaceutical compositions comprising one or more of said pharmaceutical reagents and to methods for the treatment of cancer using said pharmaceutical compositions, in particular to methods of gene therapy.

This application is a continuation-in-part of U.S. patent application Ser. No. 10/252,132 filed Sep. 19, 2002.

### FIELD OF THE INVENTION

The present invention relates to murine genomic regions, identified by retroviral insertional tagging of mice as being involved in tumor development, in particular leukemia development, as well as human homologues thereof, and to the use of these genomic regions for the identification and development of anti-cancer drugs, such as small molecule inhibitors, antibodies, ribozymes, antisense molecules and RNA interference (RNAi) molecules, that are effective in reducing or eliminating the tumorigenic effects of genetic transformations in these genomic regions and/or eliminating the tumorigenic effects of expression products thereof. The invention further relates to these anti-cancer drugs and to their use as pharmaceutical reagents for the treatment of cancer, as well as to pharmaceutical compositions comprising one or more of said pharmaceutical reagents and to methods for the treatment of cancer using said pharmaceutical compositions, in particular to methods of gene therapy. In yet further aspects, the invention relates to nucleic acids derived from said murine genomic regions involved in tumor development or fragments thereof, to antibodies raised against the expression products of genes the sequence of which is comprised in said murine genomic regions or human homologues thereof, to antibodies raised against the expression products of genes affected by genetic transformations in said murine genomic regions or human homologues thereof, to the use of said nucleic acids or antibodies as diagnostic reagents for the diagnosis of cancer, as well as to diagnostic compositions comprising one or more of said diagnostic reagents and to methods for the diagnosis of cancer using said diagnostic compositions.

### BACKGROUND OF THE INVENTION

After a quarter century of rapid advances, cancer research has generated a rich and complex body of knowledge revealing cancer to be a disease involving dynamic changes in the genome. Cancer is thought to result from six essential alterations in cell physiology that collectively dictate malignant growth: self-sufficiency in growth signals, insensitivity to growth-inhibitory (anti-growth) signals, evasion of programmed cell death (apoptosis), limitless replicative potential, sustained angiogenesis, and tissue invasion and metastasis.

In general, these essential alterations are the result of mutations in genes involved in the control of these cellular processes. These mutations include deletions, point mutations, inversions and amplifications. The mutations result in either an aberrant level, timing, and/or location of expression of the encoded protein or a change in function of the encoded protein. These alterations can affect cell physiology either directly, or indirectly, for example via signaling cascades.

The possibilities to identify genes that promote the transition from a normal cell into a malignant cell is an essential prerequisite for the development of novel therapies for the treatment of cancer.

One of the most common therapies for the treatment of cancer is chemotherapy. Herein, the patient is treated with one or more drugs that function as inhibitors of cellular growth and are therefore intrinsically toxic. Since cancer cells are among the fastest growing cells in the body, these cells are severely affected by the applied drugs. However, normal cells are also affected which results—besides toxicity—in very severe side-effects such as loss of fertility.

Another commonly used therapy to treat cancer is radiation therapy or radiotherapy. Radiotherapy uses high energy rays to damage cancer cells resulting in the induction of cell cycle arrest. Cell cycle arrest will ultimately result in programmed cell death (apoptosis). However, also normal cells are irradiated and damaged. In addition, it is difficult to completely obliterate all tumor cells using this therapy. Importantly, very small tumors and developing metastases cannot be treated by radiotherapy. Moreover, irradiation can cause mutations in the cells surrounding the tumor which increases the risk of developing new tumors. Combinations of both chemo- and radiotherapy are also frequently used and a subsequent accumulation of side-effects is observed.

The major disadvantage of both therapies is that they do not discriminate between normal and tumor cells. Furthermore, tumor cells have the tendency to become resistant to these therapies, especially to chemotherapy.

Therapies directed at tumor-specific targets would increase the efficiency of the therapy due to i) a decrease in the chance of developing drug resistance, ii) a reduced toxicity since the drugs used in these tumor-specific therapies are applied at much lower concentrations and iii) observed side-effects are reduced since only tumor cells are affected.

The use of tumor-specific therapies is limited by the number of targets known. Since tumors may arise from a large number of different and discrete changes in the genome, the genotype between tumor cells may vary considerably. The disease type that they cause may however still be classified as the same. This is one of the main reasons why not a single therapy exists that is effective in all patients with a certain type of cancer.

Pharmacogenetics and pharmacogenomics aim at determining the genetic determinants linked to diseases. Most of the disease are multigenic diseases, and the identification of the genes involved therein should allow for the discovery of new targets and the development of new drugs. Pharmacogenomics also encompasses the use of specific medications according to the genotype of the patient. This should lead to a dramatic improvement of the efficiency of the drugs.

Many physiological diseases are targeted by this novel pharmaceutical approach. One can name the autoimmune and inflammatory diseases, for example Addison's Disease, Alopecia Areata, Ankylosing Spondylitis, Behcet's Disease, Chronic Fatigue Syndrome, Crohn's Disease, Ulcerative Colitis, Inflammatory Bowel Disease, Diabetes and Multiple Sclerosis.

As stated, cancers are also believed to be multigenic diseases. Some oncogenes (for exemple ras, c-myc) and tumor suppressor genes (for example p53) have previously been identified, as well as some genetic markers for predisposition (for example the genes BRCA1 and BRCA2 for breast cancer). The identification of new genes involved in other kinds of cancer should allow for a better information of the patient and the prevention of the development of the disease itself, an improved life expectancy as already observed with breast cancer (Schrag et al. 2000. JAMA 283: 617-24).

Knowledge of the identity of genes involved in cancer development therefore greatly facilitates the development of prophylactic, therapeutic and diagnostic methods for this disease. Diagnosis of the affected genes in a certain tumor type allows for the design of therapies comprising the use of specific anti-cancer drugs, for instance, drugs directed against the proteins encoded by these genes.

Presently, only a limited number of genes involved in tumor development are known and there is a clear need for the identification of novel genes involved in tumor development to be used in the design of tumor-specific therapies and to define the genotype of a certain tumor.

In the research that led to the present invention, a number of genomic regions were identified to be involved in tumor development by proviral tagging. Proviral tagging (Berns. 1988. Arch Virol. 102:1-18; Kim et al. 2003. J Virol. 77:2056-62) is a method that uses a retrovirus to infect normal vertebrate cells. After infection, the virus integrates into the genome thereby disrupting the local organization of the genome. This integration is random and affects the expression or function of genes, depending on the integration site of the virus, which may for instance be in a coding region, a regulatory region or a region nearby a gene. If a cellular gene involved in tumor development is affected, the cell will acquire a selective advantage to develop into a tumor as compared to cells in which no genes involved in tumor development are affected. As a result, all cells within the tumor originating from the cell affected in a gene involved in tumor development will carry the same proviral integration. Through analysis of the region nearby the retroviral integration site, the affected gene can be identified. Due to the size of the genome and the total number of integration sites investigated in the present invention, a gene that is affected in two or more independent tumors investigated must provide a selective advantage and therefore contribute to tumor development. Such sites of integration are designated as common integration sites (CIS).

By using an improved method of proviral tagging the present inventors were able to identify a large number of murine genomic regions involved in the development of myeloid leukemia in mice. All such genomic regions comprised common integration sites of the virus nucleic acid and such sites were either found inside, before or after genomic sequences that coded for known or putative murine genes, or in yet unknown but defined positions within the murine genome. Many of the murine genomic regions, as defined by the presence therein of a common integration site, have so far not been reported to be involved in tumor development. Novel cancer related murine genomic regions identified in this manner are the following: Adam11, AI462175, Cd24a, Edg3, Itgp, Kcnj16, Kcnk5, Kcnn4, Ltb, Ly108, Ly6i, mouse homologue of EMILIN, Mrc1, Ninj2, Nphs1, Sema4b, Tm9sf2, and Tnfrsf17, encoding cell surface proteins; Apobec2, Btd, Cds2, Clpx, Ddx19, Ddx21, Dnmt2, Dqx1, Hdac7a, Lce-pending, Mgat1, mouse homologue of CILP, mouse homologue of NOH61, Nudel-pending, Pah, Pdil, Ppia, Prps1, Ptgds, and Vars2, encoding enzymes; Dagk4, mouse homologue of PSK, Nme2, Snf1lk and Tyki, encoding kinases; Dusp10, Inpp4a and Inpp5b, encoding phosphatases; Il16, Prg, and Scya4, encoding secreted factors; Akap7, Api5, Arfrp1, Arhgap14-pending, Cish2, Dapp1, Fabp6, Fkbp8, Fliz1-pending, Hint, Ier5, Jundp2-pending, Lmo6, Mid1, mouse homologue of AKAP13, mouse homologue of BIN2, mouse homologue of CEZANNE, mouse homologue of CHD2, mouse homologue of MBLL, mouse homologue of SLC16A10, mouse homologue of SLC16A6, mouse homologue of SLC17A5, mouse homologue of TAF5L, mouse homologue of U1SNRNPBP, mouse homologue of ZNF8, Mtap7, Myo1c, Nkx2-3, Nsf, Pcdh9, Pkig, Prdx2, Pscd1, Psmb1, Psme1, Psme2, Rgl1, Ril-pending, Sax1, Slc14a2, Slc7a1, Slc7a11, Swap 70, Txnip, and Ubl3, encoding signaling proteins; Clic3, Gtl1-13, mouse homologue of NOL5A, and Vdac2, encoding structural proteins; ABT1-pending, Ctbp1, Dermo1, Ebf, Elf4, Ldb1, mouse homologue of NR1D1, mouse homologue of ZER6, Rest, Tbp, Zfp238, Zfp287, and Zfp319, encoding proteins involved in transcriptional regulation; Lrrc2, Satb1, Slfn4, and genomic regions with the following Celera identification codes mCG10290, mCG10613, mCG11234, mCG11325, mCG11355, mCG11803, mCG11817, mCG12566, mCG12630, mCG12824, mCG13346, mCG14143, mCG14155, mCG14342, mCG15141, mCG15321, mCG16761, mCG16858, mCG17127, mCG17140, mCG17142, mCG17547, mCG17569, mCG17751, mCG17799, mCG17802, mCG17918, mCG18034, mCG1850, mCG18663, mCG18737, mCG20276, mCG20905, mCG20994, mCG21403, mCG21505, mCG21529, mCG21530, mCG21803, mCG22014, mCG22045, mCG22386, mCG2258, mCG22772, mCG23032, mCG23035, mCG23069, mCG23075, mCG23120, mCG2543, mCG2824, mCG2947, mCG3038, mCG3729, mCG3760, mCG50409, mCG50651, mCG5068, mCG5070, mCG51393, mCG52252, mCG52498, mCG53009, mCG53724, mCG55023, mCG55075, mCG55198, mCG55265, mCG55512, mCG56069, mCG56089, mCG56746, mCG57132, mCG57265, mCG57617, mCG57827, mCG58254, mCG58345, mCG5900, mCG5905, mCG59368, mCG59375, mCG59533, mCG59662, mCG59810, mCG59997, mCG60526, mCG60833, mCG61221, mCG61661, mCG61897, mCG61907, mCG61943, mCG62177, mCG62971, mCG63537, mCG63601, mCG64346, mCG64382, mCG64398, mCG65022, mCG65585, mCG65785, mCG66128, mCG66379, mCG66776, mCG66965, mCG7831, mCG7856, mCG8424, mCG9002, mCG9537, mCG9538, mCG9791, mCG9792, mCG9843, mCG9875, mCG9877, and mCG9880. These genes are also listed in Table 1 below.

### SUMMARY OF THE INVENTION

A first object of the present invention is to provide novel genes involved in tumor development for use in the design of tumor-specific therapies. This object is, in the case of therapies for humans, achieved by using the human homologues of the murine genomic regions listed in Table 1 for the development of inhibitors directed against the genes encoded or affected by these regions that are involved in tumor development and/or their expression products and by using these inhibitors for the preparation of pharmaceutical compositions for the treatment of cancer, preferably for the treatment of leukemia.

These murine genomic regions were discovered by using an improved method of proviral tagging involving a nested PCR approach as a result of which the cloning step as conventionally performed may now be omitted, resulting in a speeded up process of gene identification. Further, the improved method resulted in the identification of essentially only myeloid leukemia-related genes since the mice essentially only developed myeloid leukemia. Also the method supported the use of various murine leukemia viruses, which use surprisingly resulted in the identification of partly overlapping but otherwise essentially complementary sets of murine genomic regions involved in the development of murine myeloid leukemia. This then expands the possibilities of finding additional myeloid leukemia-related genes.

Another improvement of the method of the present invention for the identification of genomic regions involved in the development of cancer, preferably of murine leukemia, resides in the use of an improved method for determining integration sites as being common. This method also involves a powerful amplification procedure including nested PCR which is combined with a nucleic acid blotting procedure, preferably Southern, as illustrated in FIG. 2.

The method of the invention for the identification of genomic regions involved in the development of cancer comprises a first step of performing retroviral insertional mutagenesis of a subject, comprising infecting said subject with a tumor inducing retrovirus. In a preferred embodiment, said subject is a mouse, preferably said mouse is an NIH-Swiss mouse or an FVB/N mouse and said retrovirus is Graffi murine 1.4 leukemia virus (Gr-1.4) and/or Cas-BR-M murine leukemia virus (Cas-BR-M MuLV). The method of the invention comprises a subsequent step of isolating chromosomal DNA from primary tumors developed in the infected subject, such as present in e.g. spleen, liver, thymus, and lymph node tissue of mice with a virus-induced leukemia. The method of the invention comprises a subsequent step of digesting said chromosomal DNA with a restriction enzyme capable of cutting at least once in the genome of said tumor inducing retrovirus and at least once in the chromosomal DNA of said subject. Preferably, said chromosomal DNA is digested with a restriction enzyme recognizing restriction sites located at known positions within the virus LTR sequence but which will be unique within the genomic sequence in each virus insertion. Preferably, the restriction sites are separated such that a region of the chromosomal DNA of the subject flanking the known viral sequence can be identified based on the nucleotide sequence of said chromosomal DNA region, i.e. said region comprising a sufficient number of contiguous nucleotides to allow comparison thereof with a database of known sequences.

The method of the invention comprises a subsequent step of ligating the digested DNA to circular DNA, followed by amplification of the chromosomal DNA fragment flanking the known viral sequence. In a method of the present invention, PCR amplification is carried out by performing a first PCR reaction with said circular DNA using a first set of retrovirus (LTR; long terminal repeat)-specific primers followed by performing a second nested PCR with the product of said first PCR reaction using a second set of retrovirus (LTR)-specific primers.

Following the step of amplifying the chromosomal DNA fragment flanking the known viral sequence, the nucleotide sequence of said chromosomal DNA fragment is determined, optionally via a cloning step, but preferably by performing a direct sequencing reaction, the latter being possible owing to the powerful and specific amplification procedures used. The determined nucleotide sequence of said chromosomal DNA fragment is then compared to a database of known sequences in order to identify the subject's genomic region wherein the retrovirus was integrated thereby indicating the genomic region potentially involved in the development of cancer.

In order to determine whether an integration site is a common integration site, the method of the invention for the identification of genomic regions involved in the development of cancer comprises a step of designing genomic region-specific amplification primers, preferably in a nested format and said primers being capable of hybridizing to the chromosomal DNA fragment flanking the known viral sequence, the sequence of which fragment was determined as described hereinabove and which fragment represents a genomic region comprising a virus integration site. Then, RNA or DNA is isolated from tumors to be analysed for the presence of a common integration site and an amplification reaction, preferably a nested (RT-)PCR reaction, is performed with one or more of the genomic region-specific primers (also termed locus specific primers herein) and one or more of the virus-specific primers, preferably retrovirus (LTR)-specific primers. Next, the amplification products are blotted and the blot is separately hybridized with a virus-specific probe and a genomic region-specific probe. Amplification products hybridizing with both probes are considered to represent common integration sites, such common integration sites indicating the genomic region involved in the development of cancer.

It is an aspect of the present invention to use the murine genomic regions of the present invention for the development of inhibitors directed against the genes encoded or affected by these genomic regions and/or their expression products.

In one embodiment of the present invention, the inhibitors are antibodies and/or antibody derivatives directed against the expression products of genes encoded by the genomic regions or affected by genetic transformations in the genomic regions listed in Table 1. Therapeutic antibodies are for instance useful against gene expression products located on the cellular membrane and can be comprised in a pharmaceutical composition. Also, antibodies may be targeted to intracellular, e.g. cytoplasmic, gene products such as RNA's, polypeptides or enzymes, in order to modulate the activity of these products. Preferably, such antibodies are in the form of intrabodies, produced inside a cancer cell. In addition, antibodies may be used for deliverance of at least one toxic compound linked thereto to a tumor cell.

In a preferred embodiment of the present invention, the inhibitor is a small molecule capable of modulating the activity or interfering with the function of the protein expression product of the genes encoded by the genomic regions or affected by genetic transformations in the genomic regions involved in tumor development as defined herein. In addition, small molecules can also be used for deliverance of at least one linked toxic compound to a tumor cell.

On a different level of inhibition, nucleic acids can be used to block the production of proteins by destroying the mRNA transcribed from respective gene encoded by the genomic regions or affected by genetic transformations in the genomic regions. This can be achieved by antisense drugs, ribozymes or by RNA interference (RNAi). By acting at this early stage in the disease process, these drugs prevent the production of a disease-causing protein. The present invention relates to antisense drugs, such as antisense RNA and antisense oligodeoxynucleotides, ribozymes and RNAi molecules, directed against the genes encoded by the genomic regions or affected by genetic transformations in the genomic regions listed in Table 1 or transcription products thereof.

The expression level of a gene can either be decreased or increased during tumor development. Naturally, inhibitors are used when the expression levels are elevated. However, the present invention also provides for “enhancers”, to boost the expression level of a gene encoded by the genomic regions or affected by genetic transformations in the genomic regions involved in tumor development and of which the expression levels are reduced. “Enhancers” may be any chemical or biological compound known or found to increase the expression level of genes, to improve the function of an expression product of a gene or to improve or restore the expression of a dysfunctional gene.

Very suitable therapies to overcome reduced expression levels of a gene or to restore the expression of a dysfunctional gene encoded by the genomic regions or affected by genetic transformations in the genomic regions as disclosed herein include the replacement by gene therapy of the dysfunctional or affected gene or its regulatory sequences that drive the expression of said gene. The invention therefore relates further to gene therapy, in which a dysfunctional gene of a subject encoded by the genomic regions or affected by genetic transformations in the genomic regions as listed in Table 1 or a human homologue thereof or a dysfunctional regulatory sequence of a gene of a subject encoded by the genomic regions or affected by genetic transformations in the genomic regions as disclosed listed in Table 1 or a human homologue thereof is replaced by a functional counterpart, e.g. by stable integration of for instance a lentiviral vector comprising a functional gene or regulatory sequence into the genome of a subject's host cell which is a progenitor cell of the tumor cell-line of the subject and grafting of said transfected host cell into said subject.

The invention also relates to forms of gene therapy, in which the genes encoded by the genomic regions or affected by genetic transformations in the genomic regions listed in Table 1 or a human homologue thereof are i.a. used for the design of dominant-negative forms of these genes which inhibit the function of their wild-type counterparts following their directed expression from a suitable vector in a cancer cell.

Another object of the present invention is to provide a pharmaceutical composition for the treatment of cancer comprising one or more of the inhibitors, “enhancers”, replacement compounds, vectors or host cells according to the present invention as a pharmaceutical reagent or active ingredient. The composition can further comprise at least one pharmaceutical acceptable additive like for example a carrier, an emulsifier, or a conservative.

In addition, it is the object of the present invention to provide a method for treatment of subjects suffering from cancer which method comprises the administration of the pharmaceutical composition according to the invention to patients in need thereof in a therapeutically effective amount.

A further aspect of the present invention relates to the use of the murine genomic regions or as disclosed herein as well as to human homologues thereof or their gene expression products for the development of reagents for the diagnosis of cancers.

The invention also provides diagnostic compositions for diagnosing cancer, comprising the diagnostic reagents such as specific nucleic acid probes and/or specific antibodies capable of specifically binding to the murine genomic regions as disclosed herein as well as to human homologues thereof, to the transcription products of genes encoded thereby and/or to the expression products of such genes, respectively.

Additionally, the present invention relates to methods for diagnosing cancer, preferably leukemia, by using a diagnostic composition of the present invention.

In preferred aspects and embodiments of the present invention use is made of at least 2, preferably at least 4, more preferably at least 10, even more preferably at least 30 and most preferably all murine genomic regions selected from the group consisting of Adam11, Akap7, Arpgap14, Bomb, Cd24a, Cish2, Cig5, Clic3, Cra, Dermol, EMILIN, Flj20489, Galnt5, Hook, Ier5, IL16, Iprg1, Itgp, Kcnk5, lrrc2, Ltb, Mbll, Mrc1, Mtap7, Ninj2, Nr1d1, Pcdh9, Prdx2, Prps1, Pdi1, Ptgd3, Rgl1, Sardh, Scya4, Slc16A6, Swap70, Txnip, Trim46, Tnfrsf17 and Ub13 listed in Table 1. Using these genes very advantageous expression analysis in acute myeloid leukemia (AML) patients and/or diagnostic methods of leukemia may be performed as each of these genes appeared selectively up or down regulated (on the mRNA level) in one or more specific subgroups of AML. Particularly preferred is the use in aspects and embodiments of the present invention of at least 2, preferably at least 3, more preferably at least 4, and most preferably all genes of the group of murine genomic regions consisting of Cd24a, Cish2, Cra, Ltb and Prdx2 listed in Table 1.

In addition to the newly found genomic regions and genes affected by transformations in such regions andof which the involvement in the development in tumor was hitherto unknown, use in aspects and embodiments of the present invention may also be made of gene sequences known to be involved in cancer development, such as for instance p53, Notch-1, Evi-1, NF1 (Evi-2), Lck-1, Pim-1, HoxA9 (Evi-6), Fli-1, Yy1, Pps, Ptpn1 and N-Myc.

### DEFINITIONS

The term “murine genomic region” or “genomic region” as used herein indicates the site of integration of the nucleic acid of the virus used in proviral tagging of (mouse) genomic DNA. Such a viral integration site is involved in development of cancer when it is identified as a common integration site and may be located inside a known or putative gene, nearby, e.g. before or after a known or putative gene such as in a regulatory sequence thereof, or in a yet unknown but defined position within the (murine) genome. As such, a “murine genomic region” herein indicates a gene, a putative gene, a region nearby a gene or putative gene, or a region of the murine genome the function of which is hitherto unknown. Genes comprised in such genomic regions or genes of which the expression is affected by genetic transformations in such genomic regions are believed to be involved in cancer development as are the nucleotide sequences of the regions themselves and the expression products thereof.

“Expression products” may be protein and/or RNA.

A “murine genomic region-related gene” as used herein is a gene which is fully or partially comprised in a murine genomic region of the present invention or a gene affected by transformations therein, i.e. in general; a gene which function or expression is affected by a transformation in a murine genomic region of the invention.

The term “target cell” as used herein indicates a eukaryotic cell, preferably an animal (including human), more preferably a mammalian cell, including a specialized tissue cell and progenitor cell thereof, which is capable of being infected or transfected by vector of the invention, or which is the target for a diagnostic reagent of the present invention.

The term “producer cell” as used herein refers to a cell or cell-line, respectively, suitable for replication, propagation, and/or production, of a vector of the invention.

The term “host cell” as used herein generally indicates a cell used for the expression of a viral genome, or propagation of a vector or virus and, in the context of the present invention, includes both the target and producer cell.

The term “viral vector” refers to a nucleic acid construct comprising a viral genome capable of being transcribed in a host cell, which genome comprises sufficient viral genetic information to allow packaging of the viral RNA genome, in the presence of packaging components, into a viral particle capable of infecting a target cell. Infection of the target cell includes reverse transcription and integration into the target cell genome, where appropriate for particular viruses.

The term “isolated nucleic acid sequence” means a nucleic acid sequence that is free of the nucleotide sequences that flank the nucleic acid sequence in the naturally-occurring genome of the organism from which it is derived. The term therefore includes, for example, any recombinant DNA which is incorporated into a vector; into an autonomously replicating plasmid or into the genomic DNA of a prokaryote or eukaryote from which it has not been derived; or which exists as a separate molecule (e.g., an RNA, a cDNA or a genomic or cDNA fragment produced by PCR or restriction endonuclease digestion) independent of other sequences. It also includes a recombinant DNA which is part of a hybrid gene encoding additional polypeptide sequences.

As used herein, “polynucleotide”, “nucleic acid” or “oligonucleotide” includes reference to a deoxyribonucleotide or ribonucleotide polymer in either single-or double-stranded form, and unless otherwise limited, encompasses known analogues of natural nucleotides that hybridize to nucleic acids in a manner similar to naturally occurring nucleotides. In specific embodiments, the “polynucleotide”, “nucleic acid” or “oligonucleotide” can be substituted by chemical substances that can form sequence-specific interactions similar as for the natural phosphodiester “nucleic acid”. Known and preferred analogues include polymers of nucleotides with phosphorothioate or methylphosphonate liaisons, or peptide nucleic acids. Unless otherwise indicated, a particular nucleic acid sequence includes the complementary sequence thereof.

A coding sequence is “under the control” of regulatory sequences in a cell when RNA polymerase transcribes the coding sequence into mRNA encoded by the coding sequence.

The term “coding sequence” as defmed herein is a sequence which is transcribed into mRNA and optionally translated into a polypeptide when placed under the control regulatory sequences. The boundaries of the coding sequence are generally determined by a translation start codon ATG at the 5′-terminus and a translation stop codon at the 3′-terminus. A coding sequence can include, but is not limited to, RNA, DNA, cDNA, and recombinant nucleic acid sequences.

The term “regulatory sequences” is defined herein to include all components which are necessary or advantageous for expression of a coding sequence. Each regulatory sequence may be native or foreign to the coding sequence. Such regulatory sequences include, but are not limited to, a leader, a polyadenylation sequence, a propeptide sequence, a promoter, a signal sequence, and a transcription terminator. At a minimum, the regulatory sequences include a promoter, and transcriptional and translational stop signals. The regulatory sequences may be provided with linkers for the purpose of introducing specific restriction sites facilitating ligation of the regulatory sequences with the coding region of the nucleic acid sequence encoding a polypeptide.

The term “promoter” is used herein for its art-recognized meaning to denote a portion of a gene containing DNA sequences that provide for the binding of RNA polymerase and initiation of transcription. Promoter sequences are commonly, but not always, found in the 5′ non-coding regions of genes.

The term “packaging components” refers to building blocks which are necessary for encapsulation of nucleic acid of a virus into mature viral particles.

The terms “nucleotide sequence homology” or “sequence homology” or “homologous sequence” as used herein denote the presence of homology between two or more polynucleotides. Polynucleotides have “homologous” sequences if the sequence of nucleotides in their sequences is the same when aligned for maximum correspondence. Sequence comparison between two or more polynucleotides is generally performed by comparing portions of two sequences over a comparison window to identify and compare local regions of sequence similarity. The comparison window is generally from about 20 to 200 contiguous nucleotides. The “percentage of sequence homology” for polynucleotides, such as 50, 60, 70, 80, 90, 95, 98, 99 or 100 percent sequence homology may be determined by comparing two optimally aligned sequences over a comparison window, wherein the portion of the polynucleotide sequence in the comparison window may include additions or deletions (i.e. gaps) as compared to the reference sequence (which does not comprise additions or deletions) for optimal alignment of the two sequences. The percentage is calculated by: (a) determining the number of positions at which the identical nucleic acid base occurs in both sequences to yield the number of matched positions; (b) dividing the number of matched positions by the total number of positions in the window of comparison; and (c) multiplying the result by 100 to yield the percentage of sequence homology. Optimal alignment of sequences for comparison may be conducted by computerized implementations of known algorithms, or by visual inspection. Readily available sequence comparison and multiple sequence alignment algorithms are, respectively, the Basic Local Alignment Search Tool (BLAST) (Altschul, S. F. et al. 1990. J. Mol. Biol. 215:403; Altschul, S. F. et al. 1997. Nucleic Acid Res. 25:3389-3402) and ClustalW programs both available on the internet. Other suitable programs include GAP, BESTFIT and FASTA in the Wisconsin Genetics Software Package (Genetics Computer Group (GCG), Madison, Wis., USA).

As used herein, the term “substantially homologous” for nucleic acid sequences should be interpreted as two nucleic acid sequences having a “percentage of sequence homology” of at least 40, preferably at least 60, more preferably at least 80, even more preferably at least 90, still more preferably at least 95, still more preferably at least 98, and most preferably at least 99 percent nucleotide sequence homology.

As used herein, the term “human homologue” should be interpreted as a human (putative) gene having the same function as the (putative) gene identified in mouse. A “human homologue” refers with respect to its respective murine gene as described in the present invention to a human gene which encodes an amino acid sequence which shows more than 45%, preferably more than 60%, more preferably more than 70%, still more preferably more than 80%, even more preferably more than 90% and most preferably more than 95% identity with the amino acid sequence derived from the murine genes of the invention and is in the case of an enzyme the amino acid sequence of a polypeptide which shows the same type of enzymatic activity as the enzyme encoded murine genes of the invention. A “human homologue” of a murine genomic region, not being a (putative) gene, as defined herein indicates a genomic region within the human genome that, when genetically transformed, results in an aberrant function of a gene which is identical in human and mouse.

As used herein, the terms “antibody” and “antibodies” refer to monoclonal antibodies, multispecific antibodies, synthetic antibodies, human antibodies, humanized antibodies, chimeric antibodies, single-chain Fvs (scFv), single chain antibodies, Fab fragments, F(ab′) fragments, disulfide-linked Fvs (sdFv), and anti-idiotypic (anti-Id) antibodies (including, e.g., anti-Id antibodies to antibodies of the invention), and epitope-binding fragments of any of the above. In particular, antibodies of the present invention include immunoglobulin molecules and immunologically active portions of immunoglobulin molecules, i.e., molecules that contain an antigen binding site that immunospecifically binds to a polypeptide antigen encoded by a gene comprised in the genomic regions or affected by genetic transformations in the genomic regions listed in Table 1. The immunoglobulin molecules of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule.

The term “oligonucleotide array” refers to a substrate having a two-dimensional surface having at least two different features. Oligonucleotide arrays preferably are ordered so that the localization of each feature on the surface is spotted. In preferred embodiments, an array can have a density of at least five hundred, at least one thousand, at least 10 thousand, at least 100 thousand features per square cm. The substrate can be, merely by way of example, glass, silicon, quartz, polymer, plastic or metal and can have the thickness of a glass microscope slide or a glass cover slip. Substrates that are transparent to light are useful when the method of performing an assay on the chip involves optical detection. As used herein, the term also refers to a probe array and the substrate to which it is attached that form part of a wafer. The substrate can also be a membrane made of polyester or nylon. In this embodiment, the density of features per square cm is comprised between a few units to a few dozens.

“Modulating” according to the present invention should be understood as regulating, controlling, blocking, inhibiting, stimulating, enhancing, activating, mimicking, bypassing, correcting, removing, and/or substituting said gene expression or said route, in more general terms, intervening in said gene expression or said route.

“Subject” as used herein includes, but is not limited to, an organism; a mammal, including, e.g., a human, non-human primate, mouse, pig, cow, goat, cat, rabbit, rat, guinea pig, hamster, horse, monkey, sheep, or other non-human mammal; and a non-mammal, including, e.g., a non-mammalian vertebrate, such as a bird (e.g., a chicken or duck) or a fish, and a non-mammalian invertebrate.

### LEGEND TO THE FIGURES

FIG. 1 is a graphic representation of a directed PCR on chromosomal DNA to determine commonality of inverse PCR identified virus integration site as described in Example 3. Herein, primers X1, X1N, X2 and X2N are designed in a locus or genomic region that was identified as a virus integration site by inverse PCR. To amplify flanking genomic sequences and to determine the localization and orientation of the integrated provirus, 4 different nested PCRs were performed. First, the primers X1 and X2 were combined with the Graffi-1.4 MuLV LTR primers L1 and L2. These products were amplified by a nested PCR, using the primers X1N and X2N in combination with L1N and L2N. The specificity of the amplified bands was checked using probes P1 and P2 in Southern blot analysis.

FIG. 2 illustrates the method of the present invention for the identification of disease loci. IPCR or RT-PCR was performed on RNA or DNA from cell lines (DA and NFS), or CSL leukemias. The resulting virus flanking fragments were subjected to sequence analysis. LTR- and locus-specific primers were designed and used in a nested PCR strategy (*), i.e. LTR-1/primer-A PCR, followed by a LTR-2/primer-B amplification using genomic DNA from a panel of CasBr-M MuLV-induced leukemias. PCR products were electrophoresed on a 1.5% agarose gel and subsequently blotted. Blots were first hybridized with locus-specific probe C and exposed to film, and after stripping rehybridized with probe LTR3. Bands hybridizing with the LTR3 as well as the locus specific probe C (lanes 1, 2, 3, 6 and 8) were considered positive, i.e. tumors carrying this particular common viral integration site (cVIS). Hybridization with one primer only (lanes 4 and 7) are false positives. In each experiment two or three positive fragments were cloned and nucleotide sequenced to confirm specificity

### DETAILED DESCRIPTION OF THE INVENTION

As outlined above, knowledge of the identity of genes involved in cancer development greatly facilitates the development of prophylactic, therapeutic and diagnostic methods for this disease. The discovery of a great number of additional cancer genes and potential cancer genes now provides for the tools to detect and treat cancer and to provide for prophylactic, therapeutic and diagnostic compositions useful for treating subjects suffering from cancer or diagnosing subjects suspected of having the disease or even diagnosing the severity or type of the disease.

In a preferred embodiment, the present invention is aimed at providing methods, substances, compositions, and uses according to the invention for the treatment of leukemia, even more preferably for the treatment of acute myeloid leukemia (AML).

Leukemia is a type of cancer that originates in the bone marrow where the blood forming cells (hematopoietic stem cells and progenitor cells) in adults reside. Leukemia is an uncontrolled production and accumulation of cancerous blood cells that have usually partly or completely lost their ability to develop into differentiated and functional blood cell types. Leukemias can be divided in clinically distinct categories with largely different prognosis and requirements for the type of treatment. Based on clinical and cytological parameters, leukemias are grossly classified in the following categories: chronic myeloid leukemia (CML), chronic lymphocytic leukemia (CLL), acute myeloid leukemia (AML) and acute lymphoblastic leukemia (ALL). The clinico-pathological characteristics of these types of leukemia are distinct and require different forms of treatment with variable aggressiveness and treatment-related morbidity and mortality.

In the past two decades, a further refinement of the above-mentioned classification has been developed based on immunological, cytogenetic and, more recently, molecular genetic parameters. This refinement has resulted in improvements in treatment selection and a better prediction of the prognosis and treatment responses of patients and has formed the basis for the categorization of leukemia patients into good risk, standard risk and poor risk categories. However, these categories are still heterogeneous and further refinement is urgently required.

In addition, further insights in the pathogenetic and pathophysiological mechanisms underlying this heterogeneity is urgently needed as it would provide an invaluable source of information for the development of new therapies aimed at specific pathogenetic mechanisms. This will not only result in reduced mortality and morbidity with equally efficient therapeutic impact, but also in the development of curative therapies for patients that are refractory to the currently available treatment options for leukemia.

Many of the genes affected by retroviral integration, as described in more detail in the examples below, appeared to be related to signaling, some of which have already been linked to human leukemia. For instance, in case of the Graffi-1.4 MuLV, the Tie-1 gene, which encodes a tyrosine kinase receptor that is normally expressed in vascular endothelial cells and hematopoietic stem cells, is overexpressed in chronic myeloid leukemia (CML). Importantly, high Tie-1 levels inversely correlate with survival of CML patients in early chronic phase. Strongly increased levels of Tie-1 were also detected in bone marrow samples from MDS Myelodysplastic Syndromes) and AML patients. Notch-1 is another example of a CIS associated with human disease. The human homologue of the Notch (Tan-1) gene is involved in chromosomal translocation t(7; 9)(q34; 34.3) which results in a truncated receptor in human T-lymphoblastic neoplasms. Notch-1 is a proviral integration site in mouse lymphoid leukemias. Our data suggest that aberrant Notch signaling may also be involved in myeloid leukemia. Notably, the integrations in the Notch gene predictably result in the constitutive formation of the truncated active form of Notch, which interferes with G-CSF-induced myeloid differentiation in the 32D cell model.

Thus, the newly detected involvement of the gene sequences or murine genomic regions as disclosed herein, allows for the identification of novel routes of disease development and for a better understanding in the cooperative action between genes in pathogenesis of cancer, in particular of leukemia and thus for a better diagnosis and treatment of the disease.

The implications of the findings reported herein are not limited to murine cancers. Many of the murine genomic regions identified herein also play an important role in cancer development in humans and other mammals. Examples of genes that are involved in mouse as well as human leukemia are for instance the tumor suppressor gene Evi2/Nf1 (Buchberg et al., Mol Cell Biol, 10, 4658-4666, 1990; Shannon et al., N Engl J Med, 330, 597-601, 1994; Side et al., Blood, 92, 267-722, 1998), or the oncogenes Nmyc-1 (Hirvonen et al. Leuk Lymphoma, 11, 197-205, 1993; Setoguchi et al., Mol Cell Biol, 9, 4515-4522, 1989), Evil (Morishita et al. Oncogene Res, 5, 221-231, 1990; Morishita Cell, 54, 831-840, 1988), Evi6/Hoxa9 (Nakamura et al., Nat Genet, 12, 154-158, 1996; Nakamura et al., Nat Genet, 12, 149-153, 1996), Bcl1/CyclinD1 (de Boer et al., 1997 Ann Oncol, 8, 109-117; Silver & Buckler, J Virol, 60, 1156-1158,1986), Erg (Shimizu et al., Proc Natl Acad Sci USA, 90, 10280-10284 1993; Valk et al., Nucleic Acids Res, 25, 4419-4421, 1997) or spi-1/Pu.1 (Mueller B U et al., Blood 101(5):2074, 2003). An overview of currently known murine cancer genes identified in retroviral screens can be found on the internet address http://genome2.ncifcrfgov/RTCGD/. Likewise, it is therefore predicted that a large number of the genes as disclosed herein is involved in cancer development in humans as well.

Mutations are frequently observed in genes encoding growth factors, growth factor receptors and other membrane proteins, kinases, phosphatases and other regulatory enzymes, transcription regulators and proteins critical in the process of survival and apoptosis. Multiple “leukemia genes” have been demonstrated to be disease genes responsible for the development of certain other types of malignancies as well, e.g. Ras (Bos J L, et al Blood. 69:1237-41,1987;, Galiana C. et al., Mol Carcinog. 14:286-93, 1995; van 't Veer et al., Oncogene 2:157-65, 1988), p53 (Carson D A, Lois A., Lancet. 14;346(8981):1009-11, 1995). NF-1 (McLaughlin M E, Jacks T, Methods Mol Biol. 222: 223-237, 2003; Nishi T, Saya H., Cancer Metastasis Rev. 10: 301-310, 1991) or C-kit (Rubin B P et al, Cancer Res. 61 :8118-8121, 2001).

Acute myeloid leukemia (AML) is the most frequent form of acute leukemia in adults and is one of the most aggressive forms of leukemia, which is acutely life threatening unless treated with different kinds of chemotherapy. Depending on the AML subtype determined by various clinical parameters, including age, and laboratory findings, for instance cytogenetic features, allogeneic stem cell transplantation might follow the remission induction by chemotherapy. The 5 years overall and disease free survival rate of adult AML is currently in the order of 35-40%. There is a strong need for a more precise diagnosis of AML, which allows for better distinction between the prognostic subtypes and for new therapeutic strategies for the large contingent of patients that can not be cured to date. The currently available laboratory techniques allow for a prognostic classification, but this is still far from optimal. Still, most patients cannot satisfactorily be risk-stratified and still a majority of patients are not cured by currently available treatment modalities.

The pathogenesis of leukemia is complex. Before becoming clinically overt, leukemic cells have acquired multiple defects in regulatory genes that control normal blood cell production. In human leukemia, until now only few of these genes have been identified, mainly by virtue of the fact that these genes were located in critical chromosomal regions involved in specific chromosome translocations found in human AML. Studies in mice, particularly those involving retroviral tagging, have yielded only relatively small numbers of retroviral insertions and target genes per study, but have nonetheless made clear that there are at least a few hundred genes that can be involved in the pathogenesis of murine leukemia. Based on the strong conservation between the mouse and human hematopoietic systems, as is for instance evident from the fact that the biological properties of the hematopoietic progenitor cells and the regulators (hematopoietic growth factors) are largely similar, it is very likely that a substantial proportion of the genes (or their close family members) implicated in mouse leukemia, can also be involved in the development of human disease.

In order to substantiate the claim that the presently disclosed genes play an important role in human leukemia we investigated 288 cases of human AML. These consisted of AML cases with internal tandem duplications in the gene encoding the tyrosine kinase receptor FLT3 (FLT3-ITD), which feature is associated with a poor prognosis, as well as AML cases with a t(8; 21) translocation or patients with a t(15; 17) translocation, which features fall within the favourable risk categories. For 126 of the 237 genes as disclosed herein, a study for their potential involvement said 288 cases of human AML was performed. The study included the determinations of 1) whether the genes could be detected, 2) whether the expression of the genes deviated significantly from expression levels in normal human bone marrow cells, in particular the immature (CD 34+) subset, and 3) to what extent these genes might be differentially expressed in the most significant prognostic subgroups of AML that are defined to date. The results of these determinations indicated that the genes as disclosed herein provide for additional diagnostic power in detecting as well as in differentiating AML.

Although a large number of the murine genomic regions as disclosed in the present invention, i.e. in Table 1, encode known genes, i.e. encode for known murine genes, the relevance of these genes in the development of myeloid leukemia was not previously recognized.

Furthermore, the remainder of the murine genomic regions involved in tumor development as disclosed in the present invention comprise unknown nucleic acid sequences, i.e. do not show any significant homology to sequences that encode known murine genes as available in present-day databases and comprise i.a. putative gene sequences. Nonetheless, the involvement of these murine genomic regions in the development of myeloid leukemia was demonstrated for all nucleic acid sequences as listed in Table 1.

The finding that these murine genomic regions are involved in the development of myeloid leukemia does not preclude their role in other forms of leukemia or even other cancers. Therefore, the murine genomic regions as listed in Table 1 may also play an important role in cancers such as bone cancers, brain tumors, breast cancer, endocrine system cancers, gastrointestinal cancers, gynaecologic cancers, head and neck cancers, lung cancers, lymphomas, metastases, myelomas, pediatric cancers, penile cancer, prostate cancer, sarcomas, skin cancers, testicular cancer, thyroid cancer and urinary tract cancers.

The present invention provides in one aspect for nucleic acid sequences suitable for therapeutic and/or diagnostic use.

Murine genomic regions disclosed in the present invention are useful for developing therapeutic and diagnostic methods. For example, a diagnostic assay to determine the stage of the disease may be developed and may also be useful in tailoring the treatment of aggressive versus milder cancer or tumor progression. For instance it is known that the t(15; 17) translocation results in a mutation in the gene encoding a all-trans retinoic acid (ATRA) receptor in differentiating blood cells and results in an impaired ATRA receptor activity, required for proper differentiation. As a result, differentiation of the cells is impaired leading to leukemia. A well accepted treatment is currently the administration of high doses of ATRA in order to allow the cells to differentiate. This demonstrates that knowledge of the molecular mechanism of cancer may provide important implications for therapy.

The murine genomic regions, genes comprised therein and polypeptides encoded thereby as well as genes and their encoded polypeptides affected by transformations in murine genomic regions of the invention are useful for designing diagnostic reagents useful in such methods.

Further, modulation of genes or gene expression products that are mis-regulated can be used to treat or ameliorate cancer, tumor progression, hyperproliferative cell growth, and the accompanying physical and biological manifestations. For example, the murine genomic regions provided herein, can be used to construct nucleic acid and polypeptide compositions useful for treatment, such as antisense, ribozymes, antibodies, vaccine antigens, and immune system inducers, for example. Thus, the present invention relates to methods and reagents for diagnosis, and to methods and compositions for treatment.

In one aspect, use is provided of nucleic acids having a sequence of one or more of the cancer-related genes as disclosed herein to obtain full-length cDNA and full-length human gene and promoter regions. One such use is the generation of the polypeptides encoded by the gene sequence.

Polypeptide Production

The polynucleotide, the corresponding cDNA, or the full-length gene encoded in a murine genomic region of the invention may be identified by using computer algorithms for the identification of open reading frames (ORFs) therein. Subsequently, full length cDNAs comprising the complete or partial coding sequences may be prepared synthetically and ligated into a suitable expression system to yield a polynucleotide construct of the invention. Appropriate polynucleotide constructs are purified using standard recombinant DNA techniques as described in, for example, Sambrook et al., (1989) Molecular Cloning: A Laboratory Manual, 2nd ed. (Cold Spring Harbor Press, Cold Spring Harbor, N.Y.). The polypeptides encoded by the polynucleotides are expressed in any expression system, including, for example, bacterial, yeast, insect, amphibian and mammalian systems. Suitable vectors and host cells are described in U.S. Pat. No. 5,654,173.

Expression systems in bacteria include those described in Chang et al., Nature (1978) 275:615, Goeddel et al., Nature (1979) 281:544, Goeddel et al., Nucleic Acids Res. (1980) 8:4057; EP 0 036,776, U.S. Pat. No. 4,551,433, DeBoer et al., Proc. Natl. Acad. Sci. (USA) (1983) 80:21-25, and Siebenlist et al., Cell (1980) 20:269.

Expression systems in yeast include those described in Hinnen et al., Proc. Natl. Acad. Sci. (USA) (1978) 75:1929; Ito et al., J. Bacteriol. (1983) 153:163; Kurtz et al., Mol. Cell. Biol. (1986) 6:142; Kunze et al., J. Basic Microbiol. (1985) 25:141; Gleeson et al., J. Gen. Microbiol. (1986) 132:3459, Roggenkamp et al., Mol. Gen. Genet. (1986) 202:302) Das et al., J. Bacteriol. (1984) 158:1165; De Louvencourt et al., J. Bacteriol. (1983) 154:737, Van den Berg et al., Bio/Technology (1990) 8:135; Kunze et al., J. Basic Microbiol. (1985) 25:141; Cregg et al., Mol. Cell. Biol. (1985) 5:3376, U.S. Pat. Nos. 4,837,148 and 4,929,555; Beach and Nurse, Nature (1981) 300:706; Davidow et al., Curr. Genet. (1985) 10:380, Gaillardin et al., Curr. Genet. (1985) 10:49, Ballance et al., Biochem. Biophys. Res. Commun. (1983) 112:284-289; Tilburn et al., Gene (1983) 26:205-221, Yelton et al., Proc. Natl. Acad Sci. (USA) (1984) 81:1470-1474, Kelly and Hynes, EMBO J. (1985) 4:475479; EP 0 244,234, and WO 91/00357.

Expression of heterologous genes in insects is accomplished as described in U.S. Pat. No. 4,745,051, Friesen et al. (1986) “The Regulation of Baculovirus Gene Expression” in: The Molecular Biology Of Baculoviruses (W. Doerfler, ed.), EP 0 127,839, EP 0 155,476, and Vlak et al., J. Gen. Virol. (1988) 69:765-776, Miller et al., Ann. Rev. Microbiol. (1988) 42:177, Carbonell et al., Gene (1988) 73:409, Maeda et al., Nature (1985) 315:592-594, Lebacq-Verheyden et al., Mol. Cell. Biol. (1988) 8:3129; Smith et al., Proc. Natl. Acad. Sci. (USA) (1985) 82:8404, Miyajima et al., Gene (1987) 58:273; and Martin et al., DNA (1988) 7:99. Numerous baculoviral strains and variants and corresponding permissive insect host cells from hosts are described in Luckow et al., Bio/Technology (1988) 6:47-55, Miller et al., Generic Engineering (Setlow, J. K. et al. eds.), Vol. 8 (Plenum Publishing, 1986), pp. 277-279, and Maeda et al., Nature, (1985) 315:592-594.

Mammalian expression is accomplished as described in Dijkema et al., EMBO J. (1985) 4:761, Gorman et al., Proc. Natl. Acad. Sci. (USA) (1982) 79:6777, Boshart et al., Cell (1985) 41:521 and U.S. Pat. No. 4,399,216. Other features of mammalian expression are facilitated as described in Ham and Wallace, Meth. Enz. (1979) 58:44, Barnes and Sato, Anal. Biochem. (1980) 102:255, U.S. Pat. Nos. 4,767,704, 4,657,866, 4,927,762, 4,560,655, WO 90/103430, WO 87/00195, and U.S. Pat. No. RE 30,985.

Polynucleotide molecules or parts thereof comprising the murine genomic regions listed in Table 1 or comprised therein are propagated by placing the molecule in a vector. Viral and non-viral vectors are used, including plasmids. The choice of plasmid will depend on the type of cell in which propagation is desired and the purpose of propagation. Certain vectors are useful for amplifying and making large amounts of the desired DNA sequence. Other vectors are suitable for expression in cells in culture. Still other vectors are suitable for transfer and expression in cells in a whole animal or person. The choice of appropriate vector is well within the skill of the art. Many such vectors are available commercially. The polynucleotide is inserted into a vector typically by means of DNA ligase attachment to a cleaved restriction enzyme site in the vector. Alternatively, the desired nucleotide sequence may be inserted by homologous recombination in vivo. Typically this is accomplished by attaching regions of homology to the vector on the flanks of the desired nucleotide sequence. Regions of homology are added by ligation of oligonucleotides, or by polymerase chain reaction using primers comprising both the region of homology and a portion of the desired nucleotide sequence, for example.

Polynucleotides are linked to regulatory sequences as appropriate to obtain the desired expression properties. These may include promoters (attached either at the 5′ end of the sense strand or at the 3′ end of the antisense strand), enhancers, terminators, operators, repressors, and inducers. The promoters may be regulated or constitutive. In some situations it may be desirable to use conditionally active promoters, such as tissue-specific or developmental stage-specific promoters. These are linked to the desired nucleotide sequence using the techniques described above for linkage to vectors. Any techniques known in the art may be used.

When any of the above host cells, or other appropriate host cells or organisms, are used to replicate and/or express the polynucleotides or nucleic acids of the invention, the resulting replicated nucleic acid, RNA, expressed protein or polypeptide, is within the scope of the invention as a product of the host cell or organism. The product is recovered by any appropriate means known in the art.

Once the gene corresponding to a polypeptide is identified, its expression can be regulated in the cell to which the gene is native. For example, an endogenous gene of a cell can be regulated by an exogenous regulatory sequence as disclosed in U.S. Pat. No. 5,641,670, “Protein Production and Protein Delivery.”

Identification of Secreted and Membrane-Bound Polypeptides

Both secreted and membrane-bound polypeptides encoded in murine genomic regions or affected thereby in any way are of interest. For example, levels of secreted polypeptides can be assayed conveniently in body fluids, such as blood or urine. Membrane-bound polypeptides are useful for constructing vaccine antigens, for inducing an immune response, or for diagnosis of whole cells. Such antigens would comprise all or part of the extracellular region of the membrane-bound polypeptides.

Because both secreted and membrane-bound polypeptides comprise a fragment of contiguous hydrophobic amino acids, hydrophobicity predicting algorithms can be used to identify such polypeptides.

A signal sequence is usually encoded by both secreted and membrane-bound polypeptide genes to direct a polypeptide to the surface of the cell. The signal sequence usually comprises a stretch of hydrophobic residues. Such signal sequences can fold into helical structures.

Membrane-bound polypeptides typically comprise at least one transmembrane region that possesses a stretch of hydrophobic amino acids that can transverse the membrane. Some transmembrane regions also exhibit a helical structure.

Hydrophobic fragments within a polypeptide can be identified by using computer algorithms. Such algorithms include Hopp & Woods, Proc. Natl. Acad. Sci. USA 78: 3824-3828 (1981); Kyte & Doolittle, J. Mol. Biol. 157:105-132 (1982); and RAOAR algorithm, Degli Esposti et al., Eur. J. Biochem. 190: 207-219 (1990).

Another method of identifying secreted and membrane-bound polypeptides is to translate the present polynucleotides, listed in Table 1, and determine if at least 8 contiguous hydrophobic amino acids are present. Those translated polypeptides with at least 8; more typically, 10; even more typically, 12 contiguous hydrophobic amino acids are considered to be either a putative secreted or membrane bound polypeptide. Hydrophobic amino acids include alanine, glycine, histidine, isoleucine, leucine, lysine, methionine, phenylalanine, proline, threonine, tryptophan, tyrosine, and valine.

Construction of Polypeptides of the Invention and Variants Thereof

The polypeptides useful for the development of therapeutic reagents include those encoded by the nucleotide sequences of genes or putative genes as disclosed herein. These nucleotide sequences can also be encoded by nucleic acids that, by virtue of the degeneracy of the genetic code, are not identical in sequence to the disclosed nucleotide sequences. Thus, the invention includes within its scope isolated nucleic acids comprising a nucleotide sequence encoding a protein or polypeptide expressed by said isolated nucleic acid having the sequence of any one of the gene sequences listed in Table 1 or having a sequence substantially homologous thereto. Also within the scope of the invention are variants; variants of polypeptides include mutants, fragments, and fusions. Mutants can include amino acid substitutions, additions or deletions. The amino acid substitutions can be conservative amino acid substitutions or substitutions to eliminate non-essential amino acids, such as to alter a glycosylation site, a phosphorylation site or an acetylation site, or to minimize misfolding by substitution or deletion of one or more cysteine residues that are not necessary for function. Conservative amino acid substitutions are those that preserve the general charge, hydrophobicity/hydrophilicity, and/or steric bulk of the amino acid substituted. For example, substitutions between the following groups are conservative: Gly/Ala, Val/Ile/Leu, Asp/Glu, Lys/Arg, Asn/Gln, Ser/Cys, Thr, and Phe/Trp/Tyr. The genetic code can be used to select the appropriate codons to construct the corresponding variants.

Small Molecule Inhibitors

Small molecule inhibitors are usually chemical entities that can be obtained by screening of already existing libraries of compounds or by designing compounds based on the structure of the protein encoded by a gene involved in tumor development. Briefly, the structure of at least a fragment of the protein is determined by either Nuclear Magnetic Resonance or X-ray crystallography. Based on this structure, a virtual screening of compounds is performed. The selected compounds are synthesized using medicinal and/or combinatorial chemistry and thereafter analyzed for their inhibitory effect on the protein in vitro and in vivo. This step can be repeated until a compound is selected with the desired inhibitory effect. After optimization of the compound, its toxicity profile and efficacy as cancer therapeutic is tested in vivo using appropriate animal model systems.

Differentially expressed genes that do not encode membrane-bound proteins are selected as targets for the development of small molecule inhibitors. To identify putative binding sites or pockets for small molecules on the surface of the target proteins, the three-dimensional structure of those targets are determined by standard crystallization techniques (de Vos et al. 1988, Science 239:888-93; Williams et al. 2001, Nat Struct Biol 8:838-42). Additional mutational analysis may be performed to confirm the functional importance of the identified binding sites. Subsequently, Cerius2 (Molecular Simulations Inc., San Diego, Calif., USA) and Ludi/ACD (Accelrys Inc., San Diego, Calif., USA) software is used for virtual screening of small molecule libraries (Bohm. 1992 J Comp Aided Molec Design 6:61-78). The compounds identified as potential binders by these programs are synthesized by combinatorial chemistry and screened for binding affinity to the targets as well as for their inhibitory capacities of the target protein's function by standard in vitro and in vivo assays. In addition to the rational development of novel small molecules, existing small molecule compound libraries are screened using these assays to generate lead compounds. Lead compounds identified are subsequently co-crystallized with the target to obtain information on how the binding of the small molecule can be improved (Zeslawska et al. 2000, J Mol Biol 301:465-75). Based on these findings, novel compounds are designed, synthesized, tested, and co-crystallized. This optimization process is repeated for several rounds leading to the development of a high-affinity compound of the invention that successfully inhibits the function of its target protein. Finally, the toxicity of the compound is tested using standard assays (commercially available service via MDS Pharma Services, Montreal, Quebec, Canada) after which it is screened in an animal model system.

Ribozymes

Trans-cleaving catalytic RNAs (ribozymes) are RNA molecules possessing endoribonuclease activity. Ribozymes are specifically designed for a particular target, and the target message must contain a specific nucleotide sequence. They are engineered to cleave any RNA species site-specifically in the background of cellular RNA. The cleavage event renders the mRNA unstable and prevents protein expression. Importantly, ribozymes can be used to inhibit expression of a gene of unknown function for the purpose of determining its function in an in vitro or in vivo context, by detecting the phenotypic effect.

One commonly used ribozyme motif is the hammerhead, for which the substrate sequence requirements are minimal. Design of the hammerhead ribozyme is disclosed in Usman et al., Current Opin. Struct. Biol. (1996) 6:527-533. Usman also discusses the therapeutic uses of ribozymes. Ribozymes can also be prepared and used as described in Long et al., FASEB J. (1993) 7:25; Symons, Ann. Rev. Biochem. (1992) 61:641; Perrotta et al., Biochem. (1992) 31:16-17; Ojwang et al., Proc. Natl. Acad. Sci. (USA) (1992) 89:10802-10806; and U.S. Pat. No. 5,254,678. Ribozyme cleavage of HIV-I RNA is described in U.S. Pat. No. 5,144,019; methods of cleaving RNA using ribozymes is described in U.S. Pat. No. 5,116,742; and methods for increasing the specificity of ribozymes are described in U.S. Pat. No. 5,225,337 and Koizumi et al., Nucleic Acid Res. (1989) 17:7059-7071. Preparation and use of ribozyme fragments in a hammerhead structure are also described by Koizumi et al., Nucleic Acids Res. (1989) 17:7059-7071. Preparation and use of ribozyme fragments in a hairpin structure are described by Chowrira and Burke, Nucleic Acids Res. (1992) 20:2835. Ribozymes can also be made by rolling transcription as described in Daubendiek and Kool, Nat. Biotechnol. (1997) 15(3):273-277.

The hybridizing region of the ribozyme may be modified or may be prepared as a branched structure as described in Horn and Urdea, Nucleic Acids Res. (1989) 17:6959-67. The basic structure of the ribozymes may also be chemically altered in ways familiar to those skilled in the art, and chemically synthesized ribozymes can be administered as synthetic oligonucleotide derivatives modified by monomeric units. In a therapeutic context, liposome mediated delivery of ribozymes improves cellular uptake, as described in Birikh et al., Eur. J. Biochem. (1997) 245:1-16.

Therapeutic and functional genomic applications of ribozymes proceed beginning with knowledge of a portion of the coding sequence of the gene to be inhibited. Thus, for many genes, a nucleic acid sequence provides adequate sequence for constructing an effective ribozyme. A target cleavage site is selected in the target sequence, and a ribozyme is constructed based on the 5′ and 3′ nucleotide sequences that flank the cleavage site. Retroviral vectors are engineered to express monomeric and multimeric hammerhead ribozymes targeting the mRNA of the target coding sequence. These monomeric and multimeric ribozymes are tested in vitro for an ability to cleave the target mRNA. A cell line is stably transduced with the retroviral vectors expressing the ribozymes, and the transduction is confirmed by Northern blot analysis and reverse-transcription polymerase chain reaction (RT-PCR). The cells are screened for inactivation of the target mRNA by such indicators as reduction of expression of disease markers or reduction of the gene product of the target mRNA.

Antisense

Antisense polynucleotides are designed to specifically bind to RNA, resulting in the formation of RNA-DNA or RNA-RNA hybrids, with an arrest of DNA replication, reverse transcription or messenger RNA translation. Antisense polynucleotides based on a selected sequence can interfere with expression of the corresponding gene.

Antisense polynucleotides are typically generated within the cell by expression from antisense constructs that contain the antisense strand as the transcribed strand. Antisense polynucleotides will bind and/or interfere with the translation of the corresponding mRNA. As such, antisense may be used therapeutically the inhibit the expression of oncogenes.

Antisense RNA or antisense oligodeoxynucleotides (antisense ODNs) can both be used and may also be prepared in vitro synthetically or by means of recombinant DNA techniques. Both methods are well within the reach of the person skilled in the art. ODNs are smaller than complete antisense RNAs and have therefore the advantage that they can more easily enter the target cell. In order to avoid their digestion by DNAse, ODNs and antisense RNAs may be chemically modified. For targeting to the desired target cells, the molecules may be linked to ligands of receptors found on the target cells or to antibodies directed against molecules on the surface of the target cells.

Antisense therapy for a variety of cancers is in clinical phase and has been discussed extensively in the literature. Reed reviewed antisense therapy directed at the Bcl-2 gene in tumors; gene transfer-mediated overexpression of Bcl-2 in tumor cell lines conferred resistance to many types of cancer drugs. (Reed, J. C., N.C.L. (1997) 89:988-990). The potential for clinical development of antisense inhibitors of ras is discussed by Cowsert, L. M., Anti-Cancer Drug Design (1997) 12:359-371. Additional important antisense targets include leukemia (Geurtz, A. M., Anti-Cancer Drug Design (1997) 12:341-358); human C-ref kinase (Monia, B. P., Anti-Cancer Drug Design (1997) 12:327-339); and protein kinase C (McGraw et al., Anti-Cancer Drug Design (1997) 12:315-326.

Given the extensive background literature and clinical experience in antisense therapy, one skilled in the art can use selected nucleic acids of the invention as additional potential therapeutics.

RNAi

RNAi refers to the introduction of homologous double stranded RNA to specifically target the transcription product of a gene, resulting in a null or hypomorphic phenotype. RNA interference requires an initiation step and an effector step. In the first step, input double-stranded (ds) RNA is processed into nucleotide ‘guide sequences’. These may be single- or double-stranded. The guide RNAs are incorporated into a nuclease complex, called the RNA-induced silencing complex (RISC), which acts in the second effector step to destroy mRNAs that are recognized by the guide RNAs through base-pairing interactions. RNAI molecules are thus double stranded RNAs (dsRNAs) that are very potent in silencing the expression of the target gene. The invention provides dsRNAs complementary to the genes listed in Table 1.

The ability of dsRNA to suppress the expression of a gene corresponding to its own sequence is also called post-transcriptional gene silencing or PTGS. The only RNA molecules normally found in the cytoplasm of a cell are molecules of single-stranded mRNA. If the cell finds molecules of double-stranded RNA, dsRNA, it uses an enzyme to cut them into fragments containing in general 21-base pairs (about 2 turns of a double helix). The two strands of each fragment then separate enough to expose the antisense strand so that it can bind to the complementary sense sequence on a molecule of mRNA. This triggers cutting the mRNA in that region thus destroying its ability to be translated into a polypeptide. Introducing dsRNA corresponding to a particular gene will knock out the cell's endogenous expression of that gene. This can be done in particular tissues at a chosen time. A possible disadvantage of simply introducing dsRNA fragments into a cell is that gene expression is only temporarily reduced. However, a more permanent solution is provided by introducing into the cells a DNA vector that can continuously synthesize a dsRNA corresponding to the gene to be suppressed.

RNAi molecules are prepared by methods well known to the person skilled in the art. In general an isolated nucleic acid sequence comprising a nucleotide sequence which is substantially homologous to the sequence of at least one of the murine genomic regions listed in Table 1 and which is capable of forming one or more transcripts able to form a partially of fully double stranded (ds) RNA with (part of) the transcription product of said murine cancer genes will function as an RNAi molecule. The double stranded region may be in the order of between 10-250, preferably 10-100, more preferably 20-50 nucleotides in length.

RNAi molecules are preferably expressed from recombinant vectors in transduced host cells, hematopoietic stem cells being very suitable thereto.

Dominant Negative Mutations

Dominant negative mutations are readily generated for corresponding proteins that are active as multimers. A mutant polypeptide will interact with wild-type polypeptides (made from the other allele) and form a non-functional multimer. Thus, a mutation is in a substrate-binding domain, a catalytic domain, or a cellular localization domain. Preferably, the mutant polypeptide will be overproduced. Point mutations are made that have such an effect. In addition, fusion of different polypeptides of various lengths to the terminus of a protein can yield dominant negative mutants. General strategies are available for making dominant negative mutants. See Herskowitz, Nature (1987) 329:219-222. Such a technique can be used for creating a loss of function mutation, which is useful for determining the function of a protein.

Detection Probes and Amplification Primers

Polynucleotide probes and primers comprising at least 8, preferably at least 10, more preferably at least 12 contiguous nucleotides selected from the nucleotide sequence of a murine genomic region listed in Table 1 or selected from the nucleotide sequence of genes affected by transformations in said murine genomic regions or complements thereof are used for a variety of purposes, including identification of human chromosomes and determining transcription levels of these genes.

The nucleotide probes are labeled, for example, with a radioactive, fluorescent, biotinylated, or chemiluminescent label, and detected by well known methods appropriate for the particular label selected. Protocols for hybridizing nucleotide probes to preparations of metaphase chromosomes are also well known in the art. A nucleotide probe will hybridize specifically to nucleotide sequences in the chromosome preparations which are complementary to the nucleotide sequence of the probe. A probe that hybridizes specifically to a polynucleotide should provide a detection signal at least 5-, 10-, or 20-fold higher than the background hybridization provided with other unrelated sequences.

Nucleotide probes are used to detect expression of a gene corresponding to the polynucleotide. For example, in Northern blots, mRNA is separated electrophoretically and contacted with a probe. A probe is detected as hybridizing to an mRNA species of a particular size. The amount of hybridization is quantitated to determine relative amounts of expression, for example under a particular condition.

Probes are also used to detect products of amplification by polymerase chain reaction. The products of the reaction are hybridized to the probe and hybrids are detected. Probes are used for in situ hybridization to cells to detect expression or to perform chromosome painting. Probes can also be used in vivo for diagnostic detection of hybridizing sequences. Probes are typically labeled with a fluorescent or luminescent label. Other types of detectable labels may be used such as chromophores, radioactive isotopes, and enzymes.

Expression of specific mRNA can vary in different cell types and can be tissue specific. This variation of mRNA levels in different cell types can be exploited with nucleic acid probe assays to determine disease stages. For example, PCR, branched DNA probe assays, or blotting techniques utilizing nucleic acid probes substantially identical or complementary to nucleic acid sequences of murine genomic regions listed in Table 1 or of genes affected by transformations therein can determine the presence or absence of cDNA or mRNA related to the polynucleotides of the invention.

Examples of a nucleotide hybridization assay are described in Urdea et al., PCT WO92/02526 and Urdea et al., U.S. Pat. No. 5,124,246, both incorporated herein by reference. The references describe an example of a sandwich nucleotide hybridization assay.

Alternatively, the Polymerase Chain Reaction (PCR) is another means for detecting small amounts of target nucleic acids, as described in Mullis et al., Meth. Enzymol. (1987) 155:335-350; U.S. Pat. No. 4,683,195; and U.S. Pat. No. 4,683,202, all incorporated herein by reference. Two primer polynucleotides nucleotides hybridize with the target nucleic acids and are used to prime the reaction. The primers may be composed of sequence within or 3′ and 5′ to the nucleic acid sequences of murine genomic regions listed in Table 1 or of genes affected by transformations therein. Alternatively, if the primers are 3′ and 5′ to these nucleic acid sequences, they need not hybridize to them or the complements. Yet, they may for instance also hybridize to sequences flanking the nucleic acid sequences of murine genomic regions listed in Table 1 or of genes affected by transformations therein. A thermostable polymerase creates copies of target nucleic acids from the primers using the original target nucleic acids as a template. After a large amount of target nucleic acids is generated by the polymerase, it is detected by methods such as Southern blots. When using the Southern blot method, the labeled probe will hybridize to a nucleic acid sequence of a murine genomic region listed in Table 1 or of a gene affected by a transformation therein or complement.

Furthermore, mRNA or cDNA can be detected by traditional blotting techniques described in Sambrook et al., “Molecular Cloning: A Laboratory Manual” (New York, Cold Spring Harbor Laboratory, 1989). mRNA or cDNA generated from mRNA using a polymerase enzyme can be purified and separated using gel electrophoresis. The nucleic acids on the gel are then blotted onto a solid support, such as nitrocellulose. The solid support is exposed to a labeled probe and then washed to remove any unhybridized probe. Next, the duplexes containing the labeled probe are detected. Typically, the probe is labeled with radioactivity.

Additionally, mRNA or cDNA can be detected by using probe arrays. mRNA is obtained for instance from tumor tissue and cDNA is generated therefrom by reverse PCR. The cDNA is optionally labeled by incorporation of fluorescently labeled nucleotide analogues therein and is hybridized to the probe array. Expression levels are determined from the intensity of the fluorescent signal obtained from the corresponding or respective probe sites on the array.

Use of Polynucleotides to Raise Antibodies

The present invention provides in one aspect for antibodies suitable for therapeutic and/or diagnostic use.

Therapeutic antibodies include antibodies that can bind specifically to the expression products of the genes (partly) encoded in the nucleic acid sequences of murine genomic regions listed in Table 1 or of genes affected by transformations in these regions. By binding directly to the gene products, the antibodies may influence the function of their targets by, for example, in the case of proteins, steric hindrance, or by blocking at least one of the functional domains of those proteins. As such, these antibodies may be used as inhibitors of the function of the gene product. Such antibodies may for instance be generated against functionally relevant domains of the proteins and subsequently screened for their ability to interfere with the target's function using standard techniques and assays (see for instance Schwartzberg, 2001, Crit Rev Oncol Hematol 40:17-24; Herbst et al. Cancer 94:1593-611, 2002).

Alternatively, anti-RNA antibodies may for instance be useful in silencing messengers of the tumor-related genes of the present invention. In another alternative, antibodies may also be used to influence the function of their targets indirectly, for instance by binding to members of signaling pathways in order to influence the function of the targeted proteins or nucleic acids. In yet another alternative, therapeutic antibodies may carry one or more toxic compounds that exert their effect on the target or target cell by virtue of the binding of the carrying antibody thereto.

For diagnostic purposes, antibodies similar to those above, preferably those that are capable of binding to the expression products of the genes of the present invention may be used, and that are provided with detectable labels such as fluorescent, luminescent, or radio-isotope labels in order to allow the detection of the gene product. Preferably such diagnostic antibodies are targeted to proteinaceous targets present on the outer envelop of the cell, such as membrane bound target proteins. The use of antibodies for the diagnosis of specific types of cancer is known to the skilled person (Syrigos et al. 1999, Hybridoma 18:219-24).

The antibodies used in the present invention may be from any animal origin including birds and mammals (e.g., human, murine, donkey, sheep, rabbit, goat, guinea pig, camel, horse, or chicken). Preferably, the antibodies of the invention are human or humanized monoclonal antibodies. As used herein, “human” antibodies include antibodies having the amino acid sequence of a human immunoglobulin and include antibodies isolated from human immunoglobulin libraries (including, but not limited to, synthetic libraries of immunoglobulin sequences homologous to human immunoglobulin sequences) or from mice that express antibodies from human genes.

For some uses, including in vivo therapeutic or diagnostic use of antibodies in humans and in vitro detection assays, it may be preferred to use human or chimeric antibodies. Completely human antibodies are particularly desirable for therapeutic treatment of human subjects. Human antibodies can be made by a variety of methods known in the art including phage display methods described above using antibody libraries derived from human immunoglobulin sequences or synthetic sequences homologous to human immunoglobulin sequences. See also U.S. Pat. Nos. 4,444,887 and 4,716,111; and PCT publications WO 98/46645, WO 98/50433, WO 98/24893 and WO98/16654, each of which is incorporated herein by reference in its entirety.

The antibodies to be used with the methods of the invention include derivatives that are modified, i.e, by the covalent attachment of any type of molecule to the antibody such that covalent attachment. Additionally, the derivative may contain one or more non-classical amino acids.

In certain embodiments of the invention, the antibodies to be used with the invention have extended half-lives in a mammal, preferably a human, when compared to unmodified antibodies. Antibodies or antigen-binding fragments thereof having increased in vivo half-lives can be generated by techniques known to those of skill in the art (see, e.g., PCT Publication No. WO 97/34631).

In certain embodiments, antibodies to be used with the methods of the invention are single-chain antibodies. The design and construction of a single-chain antibody is described in Marasco et al, 1993, Proc Natl Acad Sci 90:7889-7893, which is incorporated herein by reference in its entirety.

In certain embodiments, the antibodies to be used with the invention bind to an intracellular epitope, i.e., are intrabodies. An intrabody comprises at least a portion of an antibody that is capable of immunospecifically binding an antigen and preferably does not contain sequences coding for its secretion. Such antibodies will bind its antigen intracellularly. In one embodiment, the intrabody comprises a single-chain Fv (“sFv”). For a review of sFv see Pluckthun in The Pharmacology of Monoclonal Antibodies, vol. 113, Rosenburg and Moore eds. Springer-Verlag, New York, pp. 269-315 (1994). In a further embodiment, the intrabody preferably does not encode an operable secretory sequence and thus remains within the cell (see generally Marasco, Wash., 1998, “Intrabodies: Basic Research and Clinical Gene Therapy Applications” Springer, N.Y.).

Generation of intrabodies is well-known to the skilled artisan and is described for example in U.S. Pat. Nos. 6,004,940; 6,072,036; 5,965,371, which are incorporated by reference in their entireties herein.

In one embodiment, intrabodies are expressed in the cytoplasm. In other embodiments, the intrabodies are localized to various intracellular locations. In such embodiments, specific localization sequences can be attached to the intranucleotide polypepetide to direct the intrabody to a specific location.

The antibodies to be used with the methods of the invention or fragments thereof can be produced by any method known in the art for the synthesis of antibodies, in particular, by chemical synthesis or preferably, by recombinant expression techniques.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Examples of phage display methods that can be used to make the antibodies of the present invention include those disclosed in WO97/13844; and U.S. Pat. Nos. 5,580,717, 5,821,047, 5,571,698, 5,780,225, and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in PCT publication No. WO 92/22324; Mullinax et al., 1992, BioTechniques 12(6):864-869 and Better et al., 1988, Science 240:1041-1043 (said references incorporated by reference in their entireties).

It is also possible to produce therapeutically useful IgG, IgA, IgM and IgE antibodies. For an overview of the technology for producing human antibodies, see Lonberg and Huszar (1995, Int. Rev. Immunol. 13:65-93). For a detailed discussion of the technology for producing human antibodies and human monoclonal antibodies and protocols for producing such antibodies, see, e.g., PCT publication No. WO 98/24893, which is incorporated by reference herein in its entirety. In addition, companies such as Medarex, Inc. (Princeton, N.J.), Abgenix, Inc. (Freemont, Calif.) and Genpharm (San Jose, Calif.) can be engaged to provide human antibodies directed against a selected antigen using technology similar to that described above.

Recombinant expression used to produce the antibodies, derivatives or analogs thereof (e.g., a heavy or light chain of an antibody of the invention or a portion thereof or a single chain antibody of the invention), requires construction of an expression vector containing a polynucleotide that encodes the antibody and the expression of said vector in a suitable host cell or even in vivo. Once a polynucl eotide encoding an antibody molecule or a heavy or light chain of an antibody, or portion thereof (preferably, but not necessarily, containing the heavy or light chain variable domain), of the invention has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, a heavy or light chain of an antibody, a heavy or light chain variable domain of an antibody or a portion thereof, or a heavy or light chain CDR, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy, the entire light chain, or both the entire heavy and light chains.

The expression vector is transferred to a host cell by conventional techniques and the transfected cells are then cultured by conventional techniques to produce an antibody of the invention. Thus, the invention includes host cells containing a polynucleotide encoding an antibody of the invention or fragments thereof, or a heavy or light chain thereof, or portion thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention (see above)

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 81:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:516-544).

Once an antibody molecule to be used with the methods of the invention has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies of the present invention or fragments thereof may be fused to heterologous polypeptide sequences described herein or otherwise known in the art to facilitate purification.

As stated above, according to a further aspect, the invention provides an antibody as defined above for use in therapy.

For therapeutic treatment, antibodies may be produced in vitro and applied to the subject in need thereof. The antibodies may be administered to a subject by any suitable route, preferably in the form of a pharmaceutical composition adapted to such a route and in a dosage which is effective for the intended treatment. Therapeutically effective dosages of the antibodies required for decreasing the rate of progress of the disease or for eliminating the disease condition can easily be determined by the skilled person.

Alternatively, antibodies may be produced by the subject itself by using in vivo antibody production methodologies as described above. Suitably, the vector used for such in vivo production is a viral vector, preferably a viral vector with a target cell selectivity for specific cancer cells.

Therefore, according to a still further aspect, the invention provides the use of an antibody as defined above in the manufacture of a medicament for use in the treatment of a mammal to achieve the said therapeutic effect. The treatment comprises the administration of the medicament in a dose sufficient to achieve the desired therapeutic effect. The treatment may comprise the repeated administration of the antibody.

According to a still further aspect, the invention provides a method of treatment of a human comprising the administration of an antibody as defined above in a dose sufficient to achieve the desired therapeutic effect. The therapeutic effect being the alleviation or prevention of various types of cancers such as bone cancers, brain tumors, breast cancer, endocrine system cancers, gastrointestinal cancers, gynaecologic cancers, head and neck cancers, leukemia, lung cancers, lymphomas, metastases, myelomas, pediatric cancers, penile cancer, prostate cancer, sarcomas, skin cancers, testicular cancer, thyroid cancer, urinary tract cancers, more in particular the remission or prevention of relapse of solid tumors of bladder, cervix, lung, colon, breast, prostate, ovary, pancreas, liver, testicle, uterus, bone, oral cavity and oropharynx tissue, preferable of leukemia, such as acute myeloid leukemia (AML), acute lymphocytic leukemia (ALL), chronic myelogenous leukemia (CML) and chronic lymphocytic leukemia (CLL), most preferably of AML.

The diagnostic and therapeutic antibodies are preferably used in their respective application for the targeting of kinases or phosphatases, which are often coupled to receptor molecules on the cell's surface. As such, antibodies capable of binding to these receptor molecules can exert their activity-modulating effect on the kinases or phosphatases by binding to the respective receptors. Also transporter proteins may be targeted with advantage for the same reason that the antibodies will be able to exert their activity-modulating effect when present extracellularly. The above targets, together with signaling molecules, represent preferred targets for the antibody uses of the invention as more effective therapy and easier diagnosis is possibly thereby.

The diagnostic antibodies can suitably be used for the qualitative and quantitative detection of gene products, preferably proteins in assays for the determination of altered levels of proteins or structural changes therein. Protein levels may for instance be determined in cells, in cell extracts, in supernatants, body fluids by for instance flow-cytometric evaluation of immunostained cancer cells. Alternatively, quantitative protein assays such as ELISA or RIA, Western blotting, and imaging technology (e.g., using confocal laser scanning microscopy) may be used in concert with the antibodies as described herein for the diagnosis of cancers, preferably leukemia.

Use of Polynucleotides to Construct Arrays for Diagnostics

A polynucleotide capable of hybridising to a murine genomic region of the present invention as well as sequences and gene products thereof is useful for determining the occurrence of cancer, tumor progression, hyperproliferative growth, and/or accompanying biological or physical manifestations. Specifically, the polynucleotides representing the murine genomic regions and genes affected by transformations therein and encoded polypeptides can be utilized to determine the occurrence of leukemia, such as AML.

To determine the occurrence of cancer, tumor progression, hyperproliferative growth, and/or accompanying biological or physical manifestations, the levels of polynucleotides and/or encoded polypeptides of the present invention in a sample are compared to the levels in a normal control of body tissues, cells, organs, or fluids. The normal control can include a pool of cells from a particular organ or tissue or tissues and/or cells from throughout the body. For such measurements either immunoassays or nucleic acid assays as known in the art can be used.

Any observed difference between the sample and normal control can indicate the occurrence of disease or disorder. Typically, if the levels of the polynucleotides and the encoded polypeptides of the present invention are higher than those found in the normal control, the results indicate the occurrence of cancer, tumor progression, hyperproliferative growth, and/or accompanying biological or physical manifestations.

In addition, the present murine genomic regions can be useful to diagnose the severity as well as the occurrence of cancer, tumor progression, hyperproliferative growth, and/or accompanying biological or physical manifestations. For example, the greater the difference observed in the sample versus the normal control of the expression products of the genes comprised in said genomic regions or affected by transformations therein, the greater the severity of the disorder, in particular, when higher levels as compared to a normal control are observed.

Polynucleotide or oligonucleotide arrays provide a high throughput technique that can assay a large number of polynucleotide sequences in a sample. This technology can be used as a diagnostic and as a tool to test for differential expression to determine function of an encoded protein. Polynucleotide or oligonucleotide arrays constitute a specific embodiment of a diagnostic composition according to the present invention.

To create arrays, polynucleotide or oligonucleotide probes are spotted onto a substrate in a two-dimensional matrix or array. Samples of polynucleotides can be labeled and then hybridized to the probes. Double stranded polynucleotides, comprising the labeled sample polynucleotides bound to probe polynucleotides or oligonucleotide, can be detected once the unbound portion of the sample is washed away.

The probe polynucleotides or oligonucleotide can be spotted on substrates including glass, nitrocellulose, etc. The probes can be bound to the substrate by either covalent bonds or by non-specific interactions, such as hydrophobic interactions. The sample polynucleotides can be labeled using radioactive labels, fluorophors, etc.

Techniques for constructing arrays and methods of using these arrays are described in EP No. 0 799 897; PCT No. WO 97/29212; PCT No. WO 97/27317; EP No. 0 785 280; PCT No. WO 97/02357; U.S. Pat. No. 5,593,839; U.S. Pat. No. 5,578,832; EP No. 0 728 520; U.S. Pat. No. 5,599,695; EP No. 0 721 016; U.S. Pat. No. 5,556,752; PCT No. WO 95/22058; and U.S. Pat. No. 5,631,734.

Further, arrays can be used to examine differential expression of genes and can be used to determine gene function. For example, arrays of the instant polynucleotide sequences of the murine genomic regions and of genes affected by transformations therein can be used to determine if any of the sequences are differentially expressed between normal cells and cancer cells, for example. High expression of a particular message in a cancer cell, which is not observed in a corresponding normal cell, can indicate a cancer specific protein.

Differential Expression

The present invention also provides a method to identify abnormal or diseased tissue in a human. The expression of a gene corresponding to a specific polynucleotide is compared between a first tissue that is suspected of being diseased and a second, normal tissue of the human. The normal tissue is any tissue of the human, especially those that express the murine genomic region-related gene including, but not limited to, blood cells, brain, thymus, testis, heart, prostate, placenta, spleen, small intestine, skeletal muscle, pancreas, and the mucosal lining of the colon.

The expression products of murine genomic region-related genes in the two tissues are compared by any means known in the art.

Murine genomic region-related mRNA in the two tissues is compared. PolyA RNA is isolated from the two tissues as is known in the art. For example, one of skill in the art can readily determine differences in the size or amount of polynucleotide-related mRNA transcripts between the two tissues using Northern blots and nucleotide probes. Increased or decreased expression of an polynucleotide-related mRNA in a tissue sample suspected of being diseased, compared with the expression of the same polynucleotide-related mRNA in a normal tissue, indicates manifestation of the disease.

Screening for Peptide Analogs and Antagonists

Polypeptide expression products of murine genomic region-related genes as well as corresponding full length genes can be used to screen peptide libraries to identify binding partners, such as receptors, from among the encoded polypeptides.

Such binding partners can be useful in treating cancer, tumor progression, hyperproliferative cell growth, and/or accompanying biological or physical manifestations. For example, peptides or other compounds that are capable of binding or interacting with membrane-bound polypeptides encoded by one or more of the murine genomic regions listed in Table 1, can be useful as a therapeutic. Also, peptides or other compounds capable of altering the conformation of any of the encoded polypeptides by one or more of the murine genomic regions listed in Table 1 can inhibit biological activity and be useful as a therapeutic.

A library of peptides may be synthesized following the methods disclosed in U.S. Pat. No. 5,010,175, and in PCT WO91/17823.

Peptide agonists or antagonists are screened using any available method, such as signal transduction, antibody binding, receptor binding, mitogenic assays, chemotaxis assays, etc. The methods described herein are presently preferred. The assay conditions ideally should resemble the conditions under which the native activity is exhibited in vivo, that is, under physiologic pH, temperature, and ionic strength. Suitable agonists or antagonists will exhibit strong inhibition or enhancement of the native activity at concentrations that do not cause toxic side effects in the subject. Agonists or antagonists that compete for binding to the native polypeptide may require concentrations equal to or greater than the native concentration, while inhibitors capable of binding irreversibly to the polypeptide may be added in concentrations on the order of the native concentration.

The end results of such screening and experimentation will be at least one novel polypeptide binding partner, such as a receptor, encoded by a murine genomic region-related gene of the invention, and at least one peptide agonist or antagonist of the novel binding partner. Such agonists and antagonists can be used to modulate receptor function in cells to which the receptor is native, or in cells that possess the receptor as a result of genetic engineering. Further, if the novel receptor shares biologically important characteristics with a known receptor, information about agonist/antagonist binding may help in developing improved agonists/antagonists of the known receptor.

Therapeutics, whether polynucleotide or polypeptide or small molecule, can be tested, for example, in the mouse tumor assay described in Pei et al., Mol. Endo. 11: 433-441 (1997).

Other models for testing polynucleotides, polypeptides, antibodies, or small molecules useful for treatment include: animal models and cell lines disclosed in Bosland, Encyclopedia of Cancer, Volume II, pages 1283 to 1296 (1997) by Academic Press. Other useful cell lines are described in Brothman, Encyclopedia of Cancer, Volume II, pages 1303 to 1313 (1997) by Academic Press

Pharmaceutical Compositions and Therapeutic Uses

Pharmaceutical compositions can comprise polypeptides, antibodies, polynucleotides (antisense, RNAi, ribozyme), or small molecules of the claimed invention, collectively called inhibitor compounds herein. The pharmaceutical compositions will comprise a therapeutically effective amount of either polypeptides, antibodies, polynucleotides or small molecules of the claimed invention.

The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature. The precise effective amount for a subject will depend upon the subject's size and health, the nature and extent of the condition, and the therapeutics or combination of therapeutics selected for administration. Thus, it is not useful to specify an exact effective amount in advance. However, the effective amount for a given situation can be determined by routine experimentation and is within the judgment of the clinician. Specifically, the compositions of the present invention can be used to treat, ameliorate, or prevent cancer, tumor progression, hyperproliferative cell growth and/or accompanying biological or physical manifestations, including leukaemia.

For purposes of the present invention, an effective dose will be from about 0.01 mg/ kg to 50 mg/kg or 0.05 mg/kg to about 10 mg/kg of the polynucleotide, polypeptide or antibody compositions in the individual to which it is administered.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which may be administered without undue toxicity. Suitable carriers may be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art.

Pharmaceutically acceptable salts can be used therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington's Pharmaceutical Sciences (Mack Pub. Co., N.J. 1991).

Pharmaceutically acceptable carriers in therapeutic compositions may contain liquids such as water, saline, glycerol and ethanol. Additionally, auxiliary substances, such as wetting or emulsifying agents, pH buffering substances, and the like, may be present in such vehicles. Typically, the therapeutic compositions are prepared as injectables, either as liquid solutions or suspensions; solid forms suitable for solution in, or suspension in, liquid vehicles prior to injection may also be prepared. Liposomes are included within the definition of a pharmaceutically acceptable carrier.

Delivery Methods

Once formulated, the pharmaceutical compositions of the invention can be (1) administered directly to the subject; (2) delivered ex vivo, to cells derived from the subject; or (3) delivered in vitro for expression of recombinant proteins.

Direct delivery of the compositions will generally be accomplished by injection, either subcutaneously, intraperitoneally, intravenously or intramuscularly, or delivered to the interstitial space of a tissue. The compositions can also be administered into a tumor or lesion. Other modes of administration include topical, oral, catheterized and pulmonary administration, suppositories, and transdermal applications, needles, and particle gunsor hyposprays. Dosage treatment may be a single dose schedule or a multiple dose schedule.

Methods for the ex vivo delivery and reimplantation of transformed cells into a subject are known in the art and described in e.g., International Publication No. WO 93/14778. Examples of cells useful in ex vivo applications include, for example, stem cells, particularly hematopoetic, lymph cells, macrophages, dendritic cells, or tumor cells.

Generally, delivery of nucleic acids for both ex vivo and in vitro applications can be accomplished by, for example, dextran-mediated transfection, calcium phosphate precipitation, polybrene mediated transfection, protoplast fusion, electroporation, encapsulation of the polynucleotide(s) in liposomes, and direct microinjection of the DNA into nuclei, all well known in the art.

Various methods are used to administer the therapeutic composition directly to a specific site in the body. For example, a small metastatic lesion is located and the therapeutic composition injected several times in several different locations within the body of tumor. Alternatively, arteries which serve a tumor are identified, and the therapeutic composition injected into such an artery, in order to deliver the composition directly into the tumor. A tumor that has a necrotic center is aspirated and the composition injected directly into the now empty center of the tumor. The antisense composition is directly administered to the surface of the tumor, for example, by topical application of the composition. X-ray imaging is used to assist in certain of the above delivery methods.

Receptor-mediated targeted delivery of therapeutic compositions containing an antisense polynucleotide, subgenomic polynucleotides, or antibodies to specific tissues is also used. Receptor-mediated DNA delivery techniques are described in, for example, Findeis et al., Trends in Biotechnol. (1993) 11:202-205; Chiou et al., (1994) Gene Therapeutics: Methods And Applications Of Direct Gene Transfer (J. A. Wolff, ed.); Wu et al., J. Biol. Chem. (1994) 269:542-46. Preferably, receptor-mediated targeted delivery of therapeutic compositions containing antibodies of the invention is used to deliver the antibodies to specific tissue.

Pharmaceutical compositions containing antisense, ribozyme or RNAi polynucleotides are administered in a range of about 100 ng to about 200 mg of polynucleotides for local administration in a gene therapy protocol. Concentration ranges of about 500 ng to about 50 mg, about 1 μg to about 2 mg, about 5 μg to about 500 μg, and about 20 μg to about 100 μg of polynucleotides can also be used during a gene therapy protocol. Factors such as method of action and efficacy of transformation and expression are considerations which will affect the dosage required for ultimate efficacy of the polynucleotides. Where greater expression is desired over a larger area of tissue, larger amounts of polynucleotides or the same amounts readministered in a successive protocol of administrations, or several administrations to different adjacent or close tissue portions of, for example, a tumor site, may be required to effect a positive therapeutic outcome. In all cases, routine experimentation in clinical trials will determine specific ranges for optimal therapeutic effect. A more complete description of gene therapy vectors, especially retroviral vectors, is contained in U.S. Ser. No. 08/869,309, which is expressly incorporated herein.

Development of Specific Therapeutic Methods

As illustrated by the case wherein a specific translocation, e.g. the t(15, 17) translocation as described above, can be assigned to a specific mutation and to a dysfunctional gene, the identity and more specifically the functionality of that gene may aid significantly in the development of methods of treatment. The functionality of a gene may be determined by methods known in the art, for instance by structure-function analysis.

Genes that are shown to be both differentially expressed and functionally important for human tumors are selected for structure-function analysis. Deletion and point mutation mutants of these genes are constructed and tested for their functional competence compared with wild-type genes (according to Ibanez et al. Structural and functional domains of the myb oncogene: requirements for nuclear transport, myeloid transformation, and colony formation. J Virol 62:1981-8, 1988; Rebay et al. Specific truncations of Drosophila Notch define dominant activated and dominant negative forms of the receptor. Cell 74:319-29, 1993). These studies lead to the identification of functionally critical domains as well as to dominant-negative variants, i.e. mutant genes that suppress the function of their endogenous counterparts (e.g. Kashles et al. A dominant negative mutation suppresses the function of normal epidermal growth factor receptors by heterodimerization. Mol Cell Biol 11:1454-63, 1991). These dominant-negatives provide additional proof for the functional importance of the genes and give insight into which therapeutic approaches can be pursued to interfere with their function or that can support their impaired function.

Development of an Inhibitor Compound

The present invention also provides methods for the development and production of inhibitor compounds capable of inhibiting the genes or expression products disclosed in the present invention that play a role in the development of cancer. Essentially, such methods are well known in the art and generally comprise a number of consecutive steps starting with the identification of genes involved in cancer, in particular as disclosed in Examples 2 and 3 below. by using retroviral insertional tagging, optionally in a specific genetic background;
b) validation of one or more of the identified genes as potential target gene(s) for the inhibitor compound by one or more of the following methods:
   confirmation of the identified gene by Northern Blot analysis in cancer cell-lines;
   determination of the expression profile of the identified gene in tumors and normal tissue;
   determination of the functional importance of the identified genes for cancer;
c) production of the expression product of the gene; and
d) use of the expression product of the gene for the production or design of an inhibitor compound.
   Functional Importance of the Identified Genes for Human Cancer

The functional importance of the differentially expressed genes for tumor cells is determined by over-expression as well as by inhibition of the expression of these genes. Selected human tumor cell lines are transfected either with plasmids encoding cDNA of the genes or with plasmids encoding RNA interference probes for the genes. RNA interference is a recently developed technique that involves introduction of double-stranded oligonucleotides designed to block expression of a specific gene (see Elbashir et al. Nature 411:494-8, 2001; Brummelkamp et al. Science 296:550-3, 2002). Using standard laboratory techniques and assays, the transfected cell lines are extensively checked for altered phenotypes that are relevant for the tumor cells, e.g. cell cycle status, proliferation, adhesion, apoptosis, invasive abilities, etc. These experiments demonstrate the functional importance of the identified genes for human cancer.

Gene Therapy

The therapeutic polynucleotides and polypeptides of the present invention may be utilized in gene delivery vehicles. The gene delivery vehicle may be of viral or non-viral origin (see generally, Jolly, Cancer Gene Therapy (1994) 1:51-64; Kimura, Human Gene Therapy (1994) 5:845-852; Connelly, Human Gene Therapy (1995) 1:185-193; and Kaplitt, Nature Genetics (1994) 6:148-153). Gene therapy vehicles for delivery of constructs including a coding sequence of a therapeutic of the invention can be administered either locally or systemically. These constructs can utilize viral or non-viral vector approaches. Expression of such coding sequences can be induced using endogenous mammalian or heterologous promoters. Expression of the coding sequence can be either constitutive or regulated.

The present invention can employ recombinant retroviruses which are constructed to carry or express a selected nucleic acid molecule of interest. Retrovirus vectors that can be employed include those described in EP 0 415 731; WO 94/03622; WO 93/25698; WO 93/25234; U.S. Pat. No. 5, 219,740; and EP 0 345 242. Preferred recombinant retroviruses include those described in WO 91/02805.

Packaging cell lines suitable for use with the above-described retroviral vector constructs may be readily prepared (see PCT publications WO 95/30763 and WO 92/05266), and used to create producer cell lines (also termed vector cell lines) for the production of recombinant vector particles. Within particularly preferred embodiments of the invention, packaging cell lines are made from human (such as HT1080 cells) or mink parent cell lines, thereby allowing production of recombinant retroviruses that can survive inactivation in human serum.

The present invention also employs alphavirus-based vectors that can function as gene delivery vehicles. Such vectors can be constructed from a wide variety of alphaviruses, including, for example, Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532). Representative examples of such vector systems include those described in U.S. Pat. Nos. 5,091,309; 5,217,879; and 5,185,440; and PCT Publication Nos. WO 92/10578; WO 94/21792; WO 95/27069; WO 95/27044; and WO 95/07994.

Gene delivery vehicles of the present invention can also employ parvovirus such as adeno-associated virus (AAV) vectors. Representative examples include the AAV vectors disclosed by Srivastava in WO 93/09239, Samulski et al., J. Vir. (1989) 63:3822-3828; Mendelson et al., Virol. (1988) 166:154-165; and Flotte et al., PNAS (1993) 90:10613-10617.

Representative examples of adenoviral vectors include those described by Berkner, Biotechniques (1988) 6:616-627; Rosenfeld et al., Science (1991) 252:431-434; WO 93/19191; Kolls et al., PNAS (1994) 91:215-219; Kass-Eisler et al., PNAS (1993) 90:11498-11502; Guzman et al., Circulation (1993) 88:2838-2848; Guzman et al., Cir. Res. (1993) 73:1202-1207; Zabner et al., Cell (1993) 75:207-216; Li et al., Hum. Gene Ther. (1993) 4:403-409; Cailaud et al., Eur. J. Neurosci. (1993) 5:1287-1291; Vincent et al., Nat. Genet. (1993) 5:130-134; Jaffe et al., Nat. Genet. (1992) 1:372-378; and Levrero et al., Gene (1991) 101:195-202. Exemplary adenoviral gene therapy vectors employable in this invention also include those described in WO 94/12649, WO 93/03769; WO 93/19191; WO 94/28938; WO 95/11984 and WO 95/00655. Administration of DNA linked to killed adenovirus as described in Curiel, Hum. Gene Ther. (1992) 3:147-154 may be employed.

Other gene delivery vehicles and methods may be employed, including polycationic condensed DNA linked or unlinked to killed adenovirus alone, for example Curiel, Hum. Gene Ther. (1992) 3:147-154; ligand linked DNA, for example see Wu, J. Biol. Chem. (1989) 264:16985-16987; eukaryotic cell delivery vehicles cells, for example see U.S. Ser. No. 08/240,030, filed May 9, 1994, and U.S. Ser. No. 08/404,796; deposition of photopolymerized hydrogel materials; hand-held gene transfer particle gun, as described in U.S. Pat. No. 5,149,655; ionizing radiation as described in U.S. Pat. No. 5,206,152 and in WO92/11033; nucleic charge neutralization or fusion with cell membranes. Additional approaches are described in Philip, Mol. Cell Biol. (1994) 14:2411-2418, and in Woffendin, Proc. Natl. Acad. Sci. (1994) 91:1581-1585.

Naked DNA may also be employed. Exemplary naked DNA introduction methods are described in WO 90/11092 and U.S. Pat. No. 5,580,859.

Further non-viral delivery suitable for use includes mechanical delivery systems such as the approach described in Woffendin et al., Proc. Natl. Acad. Sci. USA (1994) 91(24):11581-11585.

The surprisingly intricate relationship between the gene sequences as disclosed herein and the development of malignancy as unraveled by the inventors signifies the presence of novel routes of cancer development.

Due to this finding the identification of additional risk factors, novel therapeutic interventions and pharmaceuticals and the treatment and prophylaxis of cancers, and in particular of leukemia has now become available, which will ultimately result in a reduction in occurrence and/or progression of this disease as well as improved metrhods for diagnosis and prognosis.

The present invention now also provides for a method of treatment comprising modulating the expression of gene sequences as described herein.

A further object of the present invention is to provide for coordinated design and discovery of new drugs for the treatment of cancers, and in particular of leukaemia, and related diseases as well as providing compositions comprising modulators of such genes or their expression products which can serve as a basis or an ingredient of a pharmaceutical composition. The present invention therefore also relates to pharmaceutical products that comprise such modulating compositions. As a further object, the present invention provides the use of at least one modulator for the manufacture of a medicament for the treatment and/or prevention of cancers, and in particular of leukemia, and/or related disease.

The present invention will now be illustrated by way of the following non limiting examples.

### EXAMPLE 1

### Identification of Common Viral Insertion (CIS)

### INTRODUCTION

To identify common viral integration sites in mouse tumors and tumor derived cell lines, the MuLV virus was used. Mice were either infected with murine leukemia virus 1.4 (Graffi-1.4 MuLV) or with Cas-Br-M MuLV. The Graffi-MuLV is an ecotropic retroviral complex causing leukemias in mice. This viral complex does not contain oncogenic sequences itself but rather deregulates genes due to proviral integrations. Graffi-1.4 MuLV is a subclone of this complex and predominantly induces myeloid leukemias. NIH/Swiss mice infected with Cas-Br-M MuLV develop myeloid or lymphoid malignancies also as a result of retroviral insertion that affect target genes.

### MATERIALS AND METHODS

1. Induction of Leukemias

Newborn FVB/N mice or NIH/Swiss were injected subcutaneously with 100 μl of a cell culture supernatant of Graffi-1.4 MuLV or Cas-Br-M MuLV producing NIH3T3 cells, respectively. Mice were checked daily for symptoms of illness, i.e., apathy, white ears and tail, impaired interaction with cage-mates, weight-loss, and dull fur. Typically, leukemic mice suffered from enlarged spleens, livers, thymuses, and lymph nodes. From these primary tumors, chromosomal DNA was isolated for PCR-based screening. Blood samples were taken from the heart. For morphological analysis, blood smears and cytospins were fixed in methanol, May-Grünwald-Giemsa (MGG) stained and analyzed.

Single-cell suspensions of different organs were analyzed by flow cytometry using a flow cytometer. The cells were labeled with the following rat monoclonal antibodies: ER-MP54 (ER-MP54), ER-MP58 (ER-MP58), M1/70 (Mac-1), F4/80 (F4/80), RB68C5 (GR-1), ER-MP21 (transferrin receptor), TER119 (Glycophorin A), 59-AD2.2 (Thy-1), KT3 (CD3), RA3 6B2 (B220) and E13 161-7(Sca1). Immunodetection was performed utilizing a Goat-anti-Rat antibody coupled to fluorescein isothiocyanate (G_Ra-Fitc)
2. Inverse PCR on Graffi-1.4 MuLV Induced Leukemias

Genomic DNA from the primary tumors was digested with HhaI. After ligation, a first PCR was performed using Graffi-1.4 MuLV (LTR) specific primers L1 (5′ TGCAAGATGGCGTTACTGTAGCTAG 3′) (SEQ ID NO: 1) and L2 (5′ CCAGGTTGCCCCAAAGACCTG 3′) (SEQ ID NO: 2) (cycling conditions were 1 min at 94° C., 1 min at 65° C., 3 min at 720° C. [30 cycles]). For the second nested PCR the primers L1N (5′ AGCCTTATGGTGGGGTCTTTC 3′) (SEQ ID NO: 3) and L2N (5′ AAAGACCTGAAACGACCTTGC 3′) (SEQ ID NO: 4) (15 cycles) were used. The PCR reaction mixture contained 10 mM Tris-HCl (pH 8.3), 50 mM KCL, 1.5 mM MgCl₂, 200 μM dNTP's, 10 pmol of each primer and 2.5. units of Taq-polymerase. The PCR fragments were analyzed on a 1% agarose gel and cloned in a TA cloning vector according to standard procedures. PCR products were sequenced with the M13 forward primer 5′ GACCGGCAGCAAAATG 3′ (SEQ ID NO: 5) and M13 reverse primer 5′ CAGGAAACAGCTATGAC 3′ (SEQ ID NO: 6). Virus flanking genomic sequences were identified using the National Center for Biotechnology Information (NCBI) and Celera databases
3. Inverse PCR and Real-time PCR on Cas-Br-M MuLV Leukemias

Five μg of genomic DNA was digested with Sau3A or with SstI. The products were treated with T4-ligase, which resulted in the formation of circularized products. Subsequently, an inverse PCR (ICPR) strategy was used with primers specific for the Cas-Br-M MuLV LTR. For the Sau3A digested/ligated fragments, the first PCR reaction was carried out with primers pLTR4 (5′-CCG AAA CTG CTT ACC ACA-3′) (SEQ ID NO: 7) and pLTR3 (5′-GGT CTC CTC AGA GTG ATT-3′) (SEQ ID NO: 8), followed by a nested PCR using pLTR5 (5′-ACC ACA GAT ATC CTG TTT-3′) (SEQ ID NO: 9) and pLTR6 (5′-GTG ATT GAC TAC CCG CTT-3′) (SEQ ID NO: 10). Cycle conditions for both PCRs were 15″ at 94° C., 30″ at 57° C., and 2′ at 72° C. for 10 cycles, and an additional 20 cycles following the conditions 15″ at 94° C., 30″ at 570° C., and 2′30″ at 72° C. Reactions were performed using Expand High Fidelity PCR System. In case of SstI digested genomic DNA, the circularized DNA was amplified using primers pLTR9 (5′-GAC TCA GTC TAT CGG AGG AC-3′) (SEQ ID NO: 11) and pLTR1 (5′-CTT GCT GTT GCA TCC GAC TGG-3′) (SEQ ID NO: 12), and pLTR10 (5′-GTG AGG GGT TGT GTG CTC-3′) (SEQ ID NO: 13) and pLTR2 (5′-GTC TCG CTG TTC CTT GGG AGG-3′) (SEQ ID NO: 14), respectively. The first PCR was performed for 30 cycles 30″ at 94° C., 1′ at 60° C., and 3′ at 72° C. The reaction was carried out with Expand High Fidelity PCR system. Nested PCR conditions were 30 cycles of 30″ at 94° C., 1′ at 58° C., and 1′ at 72° C. This reaction was performed with Taq polymerase.

For RT-PCR, total RNA was isolated through a CsCl gradient. First strand cDNA was obtained by reverse transcriptase (RT) reactions with an oligo(dT)-adapter primer (5′-GTC GCG AAT TCG TCG ACG CG(dT)₁₅-3′) (SEQ ID NO: 15) at 37° C. with 5 μg RNA, using the Superscript™ Preamplification System. Subsequently, PCRs (1′ at 94° C., 1′ at 58° C., 3′ at 72° C. (30 cycles)) were performed on the RT reactions of the leukemias by using the LTR specific primer pLTR6 and the adapter primer (5′-GTC GCG AAT TCG TCG ACG CG-3′) (SEQ ID NO: 15). PCR products were directly cloned into pCR2.1 and subjected to nucleotide sequencing with the M13 forward primer 5′ GACCGGCAGCAAAATG 3′ (SEQ ID NO: 5) and m13 reverse primer 5′ CAGGAAACAGCTATGAC 3′ (SEQ ID NO: 6). Nucleotide sequences were compared to the NCBI and Celera databases for analysis.

Results

1. Graffi-1.4 MuLV Induced Leukemias

Leukemias developed 4 to 6 months after subcutaneous injection of newborn FVB/N mice with Graffi-1.4 MuLV. Forty-eight of the 59 leukemias (81%) analyzed exhibited morphological characteristics of myeloid cells. Blast cell percentages in the bone marrow ranged from 24 to 90% with an average of 48%. Leukemia cells expressed immunophenotypic marker profiles consistent with their myeloid appearance, e.g., ER-MP54+, ER-MP58+, CD3−, GR-1+. Six leukemias with blast-like morphology showed no immunophenotypic differentiation markers, suggesting that these tumors represented very immature leukemias. Only 3 leukemias were of T-lymphoid origin (CD3+/MP58−/Thy1+) and 2 showed mixed myeloid and erythroid features (Ter119+/ER-MP58+/F4/80+). These results demonstrate that Graffi-1.4 MuLV infection predominantly induce myeloid leukemia in FVB/N mice.

The provirus flanks were cloned, subjected to nucleotide sequencing, and blasted against the Celera and NCBI databases resulting in the identification of common insertion sites. Of the genes identified, the ones that were so far not described to be involved in tumor development are listed in Table 1 combined with the novel cancer genes identified from Cas-Br-M MuLV induced leukemias.
2. Cas-Br-M MuLV Induced Leukemias

Cas-Br-M MuLV injected newborn NIH/Swiss mice developed leukemias by approximately 140 to 400 days postinoculation. Most of these were myeloid leukemias (59%), although T-cell (21%), and mixed T-cell/myeloid (21%) leukemias were found.

To clone viral integration sites, a virus-LTR (long terminal repeat) specific inverse PCR as well as RT-PCR were applied as complementary approaches using DNA or RNA from 35 myeloid leukemias. The inverse PCR method was carried out on 19 primary leukemias and 9 cell lines, whereas the RT-PCR based technique was performed on 12 cell lines and 2 primary leukemias.

The provirus flanks were subjected to nucleotide sequencing, blasted against the Celera and NCBI databases resulting in the identification of common insertion sites. Of the genes identified, the ones that were so far not described to be involved in tumor development are listed in Table 1 combined with the novel cancer genes identified from Graffi-1.4 MuLV induced leukemias.

### DISCUSSION

Although proviral integrations occur randomly, they may affect the expression or function of nearby genes. If a gene is affected in two or more independent tumors, this indicates that these integrations provide a selective advantage and therefore contribute to tumor development. Multiple of these common insertion sites were identified of which a large number are demonstrated for the first time to play a role in cancer. Importantly, several of the other genes identified are well-known cancer genes validating the approach. This example shows that the pursued strategy can be successfully used to identify novel genes that are involved in tumor development.

### EXAMPLE 2

### Definition of Novel Routes for Development of AML

### MATERIAL AND METHODS

Graffi-1.4 MuLV-induced leukemias

Newborn mice were injected subcutaneously with 100 μl of cell culture supernatant of Graffi1.4-MuLV producing NIH3T3 cells (a gift from Dr. E. Rassart, Department des Siences Biologiques, Universite du Quebec a Montreal, Montreal, Quebec, Canada). Mice were treated and analyzed for the development of leukemia as previously described (Erkeland, et al., 2003, Blood 101:1111-7). Chromosomal DNA was isolated from the leukemic cells for PCR-based screening (Erkeland, et al., 2003, Blood 101:1111-7).

Cytological Analysis and Immunophenotyping of the Leukemic Cells

For morphological analysis, blood smears and cytospins were fixed in methanol, stained with May-Gruinwald-Giemsa, and examined using a Zeiss Axioscope microscope (Carl Zeiss BV, Weesp, The Netherlands). Single-cell suspensions of different organs were analyzed by flow cytometry using a FACScan flow cytometer (3ecton Dickinson aid Co, Mountain View, Calif., USA). Cells were labeled as described previously (Joosten et al., 2000. Virology 268:308-18) with the following rat monoclonal antibodies: ER-MP54, ER-MP58, M1/70 (Mac-1), F4/80, RB68C5 (GR-1), ER-MP21 (transferrin receptor), TER119 (Glycophorin A), 59-AD2.2 (Thy-1), KT3 (CD3), RA3 6B2 (B220) and E13 161-7 (Sca1). Immunodetection was performed using goat-anti-rat antibodies coupled to fluorescein isothiocyanate (GαRa-FITC) (Nordic, Tilburg, The Netherlands).

Inverse PCR on Graffi-1.4 MuLV Induced Leukemias

Genomic DNA from the primary tumors was digested with HhaI (CGCG). After circularization by ligation (Rapid ligation kit, Roche Diagnostics, Mannheim, Germany), a first PCR was performed using Graffi-1.4 MuLV (LTR) specific primers L1 and L2 as described in Example 1. Cycling conditions were 1 min at 94° C., 1 min at 62° C. and 3 min at 72° C. for 30 cycles. For the second nested PCR, the primers L1N and L2N as described in Example 1 (15 cycles) were used. The PCR reaction mixture contained 10 mM Tris-HCl (pH 8.3), 50 mM KCL, 1.5 mM MgCl₂, 200 μM dNTP's, 10 pmol of each primer and 2.5 units of Taq-polymerase (Pharmacia, Uppsala, Sweden). The PCR fragments were analyzed on a 1% agarose gel.

Detection of Virus Integrations by Specific Nested PCR

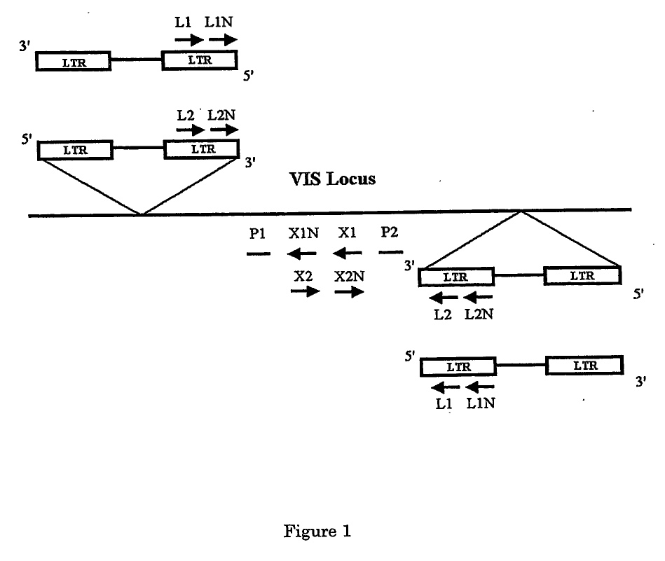

To determine the localization of the Graffi-1.4 provirus in particular virus-targeted genes in an extended panel of leukemias, a nested PCR was performed on DNA from primary tumors. For the first PCR, virus integration site (VIS) locus specific primers X1 and X2 were used in combination with Graffi-1.4 MuLV LTR specific primers L1 and L2 (see also FIG. 1). Cycling conditions were 1 min 94° C., 1 min 62° C., 3 min 72° C. for 30 cycles. For the second PCR, nested VIS-specific primers X1N and X2N (i.e. genomic region specific primers) were used in combination with nested LTR specific primers L1N and L2N under the same conditions. The obtained PCR products were analyzed by Southern blotting. To verify the correct nature of the amplified bands, the blots were hybridized with radiolabeled gene specific probes P1 and P2 at 45° C. in Church buffer (0.5 M phosphate buffer, pH 7.2, 7% (w/v) SDS, 10 mM EDTA) overnight. Signals were visualized by autoradiography according to standard procedures (Maniatis et al., 1982, Molecular Cloning, A Laboratory Manual. Cold Spring Harbor Laboratory, Cold Spring Harbor, N.Y.).

Nucleotide Sequence Analysis

PCR products were sequenced using an ABI 3100 sequencer (Applied Biosystems, Nieuwerkerk a/d IJssel, The Netherlands) with the Graffi-1.4 MuLV specific forward primer L1. Virus flanking genomic sequences were analyzed using GenBank (National Center for Biotechnology Information), Celera Discovery System (Celera Genomics, Rockville, Md., USA) (Hogenesch et al., 2001, Cell 106:413-5; Kerlavage et al., 2002, Nucleic Acids Res. 30:129-36) and Ensembl (Wellcome Trust Genome Campus, Hinxton, Cambridgeshire, UK) (Hubbard et al., 2002, Nucleic Acids Res. 30:38-41).

Results

Graffi-1.4-induced Leukemia

Eighty-nine newborn FVB/N mice were inoculated with Graffi-1.4. When moribund, mice were sacrificed and hematopoietic organs were isolated. Six mice died without signs of leukemia and were excluded from further investigation. Standard blood cell analysis was performed and values compared with the mean of 10 normal FVB/N mice (Joosten et al., 2002, Exp. Hematol. 30:142-9). Most of the leukemic mice had increased numbers of peripheral white blood cells and decreased numbers of platelets and red blood cells compared to normal controls (data not shown). Blast percentages in the bone marrow ranged from 24 to 90% with an average of 48%. Leukemic cells from 76 mice were immunophenotyped. The major immunophenotypic features of these leukemias are given in Table 2.

TABLE 2

| | | | No. of |
|---|---|---|---|
| | Leukemia type | Immunophenotype^{a} | leukemias |
| I | T-lymphoid | MP21⁺, CD3⁺, Thy1⁺ | 2 |
| | Markers | | |
| II | Mixed lymphoid, | Gr1⁺, F4/80⁺, Mac1⁺, Imm⁺(a), | 12 |
| | erythroid, myeloid | CD3⁺, B220⁺, (gcsfr⁺)(b), | |
| | differentiation | (Ter119⁺) | |
| | markers | | |
| III | Myeloid | Imm⁺, MP21⁺, (f4/80⁺, Gr1⁺, | 43 |
| | differentiation | B220⁺, Mac1⁺, gcsfr⁺) | |
| | Markers | | |
| IV | myelo-monocytic | Imm⁺, Gr1⁻, gcsfr⁻, (F4/80⁺), | 15 |
| | blasts | (Mac1+), (B220⁺) | |
| V | Erythroid | Ter119⁺, MP21⁺ (Sca1⁺) | 4 |
| Total | | | 76 |

^{a}(Imm⁺) indicates positive staining for immature hematopoietic cell markers Sca1⁺, MP58⁺, MP54⁺, (Thy1⁺).
bMarkers between brackets are not consistently expressed on all individual tumors.

Based on these criteria, the leukemias were classified as T-lymphoid, mixed lymphoid/erythroid/myeloid, myeloid, myelo-monocytic or erythroid. Fifty-nine leukemias were analyzed morphologically. Almost all of the mice showed splenomegaly of which 25% thymus enlargement, 20% with lymph node enlargement and 55% liver involvement. In 5% of the mice, leukemic cells were present in the BM and blood, without overt peripheral organ involvement.

Virus Integration Sites in Gr-1.4 Induced Leukemias

PCR analysis in conjunction with database searches identified a total of 94 different virus integration sites out of 69 tumors, 38 of which had been found previously in other studies. Examples of this latter group of CIS are p53, Notch-1, Evi-1, NF1 (Evi-2), Lck-1, Pim-1, HoxA9 (Evi-6), Fli-1 and N-Myc. Notably, 79 of the 94 integrations are CIS directly implicating the affected genes in leukemic transformation. The remaining 15 have been included in this report because closely related family members of these genes were found in other studies. Fifty-six of the identified sequences were mapped near or in novel candidate leukemia genes. The products of the affected genes have been classified in the following categories: receptors and signaling molecules (Table 3), regulators of transcription (Table 4) and the remaining group of gene products with regulatory roles in other pathways (e.g., DNA stability and proteasomal targeting) or with currently unknown functions (Table 5).

Approximately 15% of the virus integrations identified in this inverse PCR-based screen were determined to be common (more than 2 integrations in a particular genomic locus) in the initial screen. To determine the frequency of common integrations more sensitively, we performed integration specific PCR reactions on 49 Gr-1.4 target genes (tables 3-5) as described elsewhere (Erkeland, et al., 2003, Blood 101:1111-7; Joosten et al., 2002, Oncogene 21:7247-55) of which most (96%) appeared to be common. The genes flanking the virus integration sites were compared with data from the National Cancer Institute retroviral tagged cancer gene database (RTCGD) at http://genome2.ncifcrf.gov and other sources (Table 3). The Gr-1.4 integrations that are present in this database or were reported in other studies have been marked with a reference in Tables 3, 4 and 5. Family members of Gr-1.4 targeted genes found in other studies are listed in the column “identified family member”. Most of the virus integrations occurred in or near 5′ or 3′ ends of the genes, suggesting that expression levels of these genes are deregulated as a consequence of virus integration. Eighteen CIS were exclusively located within the gene, conceivably causing gene disruption. This group comprises the previously reported virus targets Notch-1, NF1, PTPN1, Inpp4a, p53, SWAP70 and Kcnk5 and 11 new genes: calcyphosine, phosphodiesterase-1, ELL, NCOR-1, HDAC-7A, histone H3.3A, api-5, RIL, D6Mm5e and two unknown genes.

Discussion

In this study, we have employed in vivo retroviral mutagenesis using the Graffi-1.4 (Gr-1.4) virus complex to identify novel routes for the pathogenesis of acute myeloid leukemia (AML) by identilying the disease genes specifically involved therein. In comparable studies with other virus strains, e.g., Moloney, AKXD or Cas-Br-M, the most prominently appearing tumor types are T and B cell lymphomas and in case of BXH2 myelomonocytic tumors. In contrast, more than 80% of the leukemias induced by Gr-1.4 unequivocally exhibited immunophenotypic characteristics of myeloid cells, with immature morphological features (mainly myeloblasts), emphasizing the unique features of the Gr-1.4 virus complex as a tool for characterizing pathogenetic mechanisms in myeloid disease (see table 2).

Indeed, the majority of the CIS described here have thus far not been reported in the extensive screens in the lymphoma models, although some overlap was observed. The latter is not surprising in view of the fact that cell type specific events usually impinge on downstream common regulatory pathways that can be affected in multiple tumor types.

Although most of the Gr-1.4 CIS have been linked to candidate disease genes based on their proximity to these genes, proviral integrations have been reported to influence gene expression over distances of more than 100 kb. Thus, it cannot be excluded that genes located more distantly from the CIS are also deregulated and that multiple genes are affected by a single CIS. In addition, three other aspects of the retroviral screens as they are currently being performed must be emphasized. First, malignancies induced by replication competent retroviruses are usually oligo- or polyclonal rather than monoclonal. Although this gives rise to high frequencies of CIS in relatively small cohorts of mice, it complicates the search for cooperating events within one leukemic clone. Second, the sensitive PCR-based techniques used to identify virus integrations do not allow distinction between CIS present in a majority of the leukemic cells, initiating an early pathogenic event, and CIS present in only a minor population of cells, probably affecting leukemia progression genes. Finally, a recent study has emphasized that murine leukemia viruses have a preference for integration near transcriptional start sites (Wu, et al., 2003, Science 300:1749-51). Our present data with Gr-1.4 corroborate this conclusion and indicate that retroviral mutagenesis with MuLV preferentially, although not exclusively, identifies gene deregulation rather than gene disruption.

In conclusion, using high-throughput retroviral screens with the Gr-1.4 virus complex we have identified novel pathways involved in myeloid leukemia. Currently, we are studying the consequences.of aberrant gene expression or function employing gene transfer methodology in 32D cells and primary bone marrow cultures.

Conclusion

Acute myeloid leukemia (AML) is a heterogeneous group of diseases, in which chromosomal aberrations, small insertions/deletions, or point mutations in certain genes have profound consequences for prognosis. However, the majority of AML patients present without currently known genetic defects. Retroviral insertion mutagenesis in mice has become a powerful tool to identify new disease genes involved in the pathogenesis of leukemia and lymphoma. Here we have used the Graffi-1.4 murine leukemia virus (Gr-1.4) strain, which causes predominantly acute myeloid leukemias, in a screen to identify novel genes involved in the pathogenesis of this disease. We report 79 candidate disease genes in common virus integration sites (CIS) and 15 genes of which family members were previously found to be affected in other studies. The majority of the identified sequences (60%) have not been found in previous screens in lymphomas and monocytic leukemias, suggesting a specific involvement in AML. Although most of the virus integrations occurred in or near the 5′ or 3′ ends of the genes, suggesting deregulation of gene expression as a consequence of virus integration, 18 CIS were exclusively located within the gene, conceivably causing gene disruption.

### EXAMPLE 3

### Large Scale Identification of Novel Potential Disease Loci in Mouse Leukemia

### MATERIAL AND METHODS

Cas-Br-M MuLV-induced Leukemias

The NIH-Swiss derived Cas-Br-M MuLV-induced primary tumors used in this study are: for IPCR: CSL 13, 16, 20, 22, 26, 30, 31, 32, 33, 65, 71, 78, 82, 90, 91, 93, 111, 117, 123, 201, 203, 204, 212, 221, 227, 228, 237 and 239; and for RT-PCR: CSL: 201 and 203 (Joosten et al. 2000. Virology 268:308-18). Cells isolated from leukemic spleens were stored vitally in aliquots in liquid nitrogen. The following leukemia cell lines were utilized in this study: for IPCR: DA 24, and NFS 22, 36, 56, 58, 60, 61, 78, and 124; and for RT-PCR: DA 1, 2, 3, 8, 33, and NFS 22, 36, 58, 61, 78, 107 and 124 (Valk et al., 1997 Blood 90:1448-1457). These cell lines were cultured in RPMI plus 10% fetal calves' serum (FCS) (Gibco Life Technologies Inc., Gent, Belgium) and 10 Units of mouse IL3. The frequency of virus integrations was determined both on cell lines, and primary leukemias.

IPCR and RT-PCR

Isolation of genomic DNA was carried out exactly as described previously (Valk et al., 1999 J. Virol. 73:3595-602). Five pg of genomic DNA was digested with Sau3A, Pvull or Sstl (GIBCO Life Technologies Inc., Gent, Belgium). The products were treated with T4-ligase (GIBCO Life Technologies Inc.), which resulted in the formation of circularized products. Subsequently we performed an IPCR strategy using primers specific for the Cas-Br-M MuLV LTR. For the Sau3A digested/ligated fragments, the first PCR reaction was carried out with primers pLTR4 (5′-CCGAAACTGCTTACCACA-3′) (SEQ ID NO. 7) and pLTR3 (5′-GGTCTCCTCAGAGTGATT-3′) (SEQ ID NO. 8), followed by a nested PCR using pLTR5 (5′-ACCACAGATATCCTGTTT-3′) (SEQ ID NO. 9) and pLTR6 (5′-GTGATTGACTACCCGCTT-3′) (SEQ ID NO. 10). Cycle conditions for both PCRs were 15 s at 94° C., 30 s at 57° C., and 2 min at 72° C. for 10 cycles, and an additional 20 cycles following the conditions 15 s at 94° C., 30 s at 57° C., and 2 min 30 s at 72° C. Reactions were performed using Expand High Fidelity PCR System (Roche, Mannheim, Germany). For PvuII and SstI digested genomic DNA, the circularized DNA was amplified using primers pLTR7 (5′-GACTCAGTCTATCGGAGGAC-3′) (SEQ ID NO. 11) and pLTRl (5′-CTTGCTGTTGCATCCGACTGG-3′) (SEQ ID NO. 12), and pLTR8 (5′-GTGAGGGGTTGTGTGCTC-3′) (SEQ ID NO. 13) and pLTR2 (5′-GTCTCGCTGTTCCTTGGGAGG-3′) (SEQ ID NO. 14) respectively.

The first PCR was performed for 30 cycles 30 s at 94° C., 1 min at 54° C., and 3 min at 72° C. Reactions were carried out with Expand High Fidelity PCR system (Roche). Nested PCR conditions were 30 cycles of 30 s at 94° C., 1 min at 58° C., and 1 min at 72° C. This reaction was performed with Taq polymerase (Amersham Pharmacia Biotech, Roosendaal, The Netherlands). RT-PCR was carried out as described previously by Valk et al. (1997c). Briefiy, total RNA was isolated through a CsCl gradient. First strand cDNA was obtained by reverse transcriptase (RT) reactions with an oligo(dT)-adapter primer (5′-GTCGCGAATTCGTCGACGCG(dT)₁₅-3′) (SEQ ID NO. 15) at 37° C. with 5 μg RNA, using the Superscript M Preamplification System (Life Technologies, Breda, The Netherlands) according to the instructions of the manufacturer. Subsequently, PCRs (1 min at 94° C., 1 min at 58° C., 3 min at 72° C. (30 cycles)) were performed on the RT reactions of the leukemias by using the LTR specific primer pLTR6 and the adapter primer (5′-GTCGCGAATTCGTCGACGCG-3′) (SEQ ID NO. 15). PCR products were directly cloned into pCR2.1 (Invitrogen, Breda, The Netherlands) according to the instructions of the manufacturer and subjected to nucleotide sequencing. Nucleotide sequences were compared to the NCBI database for analysis.

PCR Analysis and Southern Blot Hybridization

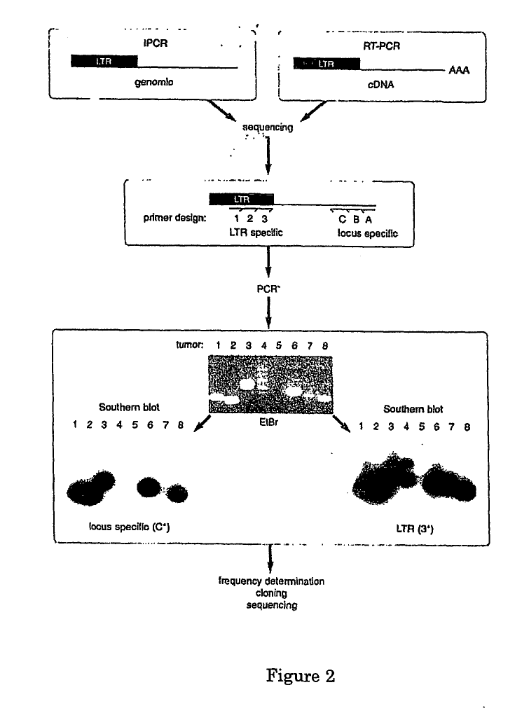

One ug of genomic DNA isolated from primary leukemias or cell lines was subjected to PCR with primer pLTRl and a locus specific primer A (FIG. 2). Locus specific primers of 17-21 nucleotides were designed specific for each of the sequences of the fragments generated by Inverse- or RT-PCR. Primers were purchased from Life Technologies. One ll of PCR product was transferred to a nested PCR reaction using primer pLTR2 and a nested locus specific primer B. Cycle conditions for both reactions were 1 cycle 5 min at 94° C., 30 cycles 30 s at 94° C., 1 min Tm, 1 min 30 s at 72° C., 1 cycle 5 min at 72° C. Tm was specific for each primer and was between 48° C. and 62° C. PCR was carried out using Taq polymerase (Amersham Pharmacia Biotech). PCR products were electrophoresed in a 1.5% agarose gel and transferred to Hybond-N⁺ nylon membranes (Amersham Pharmacia Biotech) with 0.25 M NaOH/1.5 M NaCl. Membranes were hybridized with a ³²P-end-labeled locus specific primer C. Labeling was carried out using T₄kinase (USB, Cleveland, Ohio, USA) according to the instructions of the provider. Subsequently, blots were stripped in 0.4 M NaOH for 30 min at 45° C., neutralized using 0.2 M Tris-HCl, 0.2% SDS, and 0.1×SSC for 15 min at 45° C. and hybridized with ³²P-end-labeled Cas-Br-M MuLV specific primer pLTR3. Blots were exposed for autoradiography with a KODAK film and an intensifying screen. After 15 min of exposure films were developed and analysed.

Sequencing Analysis

Samples were prepared using the Bd-sequencing kit according to instructions from the provider (PE Biosystems, Nieuwerkerk a/d IJssel, The Netherlands) and nucleotide sequencing was carried out on an ABI 310 automatic sequencer (PE Biosystems) using primers M13 forward (5′-GTAAAACGACGGCCAGT-3′) (SEQ ID NO. 24) and M13 reverse (5′-GGAAACAGCTATGACCATG-3′) (SEQ ID NO. 25). Sequences isolated by IPCR or RT-PCR were compared to the data present in the Celera Discovery System (Celera Genomics, Rockville, Md., USA, database contents May 2002) and in the GenBank of the National Center for Biotechnology Information (NCBI, Bethesda, Md., USA). The exact site of integration was determined. For insertions found outside a gene, the distance between the integration and the most nearby gene was calculated. The location on the mouse chromosome was established and the human equivalent was deduced by using the human databases of Celera Discovery System (May 2002), LocusLink or human mouse homology maps (both www.ncbi.nlm.nih.gov).

Results

Applying virus LTR-specific inverse-PCR and RT-PCR combined with automated sequencing on CasBR-M MuLV induced myeloid leukemias, 126 virus integration sites were cloned. Using locus- and LTR-specific primers, a nested PCR/Southern-blotting procedure was developed on genomic DNA from a large panel of MuLV-induced leukemias, to analyse whether a particular virus insertion represented a common virus integration site (cVIS). In fact 39 out of 41 integrations analysed this way represented a common viral integration site. We recognized six previously cloned cVISs, i.e. Evi1, Hoxa7, cMyb, Cb2/Evi11, Evi12, and His1 and 33 novel common insertions. Among this group we found integrations in or near genes encoding nuclear proteins, e.g. Dnmt-2, Nm23-M2, Ctbp1 or Erg, within receptor genes, e.g. Cb2 or mrc1, novel putative signaling or transporter genes, the ringfinger-protein gene Mid1 and a panel of genes encoding novel proteins with unknown function. The finding that 39 out of 41 integrations analysed represented a cVIS, suggests that the majority of the other virus insertions that were not yet analysed by the PCR/Southern-blotting method are located in e cVIS as well and may therefore also harbor novel disease genes.

TABLE 1

| | | | | Virus integrations in loci | | | |
|---|---|---|---|---|---|---|---|
| | | | | with putative genes or in | | | |
| Celera Gene | Mouse Gene | | Virus integrations in or | unknown genomic | Human Gene | | |
| Symbol | Symbol | Mouse Gene Name | near novel genes | regions | Symbol | Human Gene Name | Group |
| mCG7457 | Adam11 | A disintegrin and | | | ADAM11 | A disintegrin and | cell-surface |
| | | metalloprotease domain 11 | | | | metalloprotease domain 11 | |
| mCG7897 | AI462175 | Expressed sequence AI462175 | | | SMAP1 | Stromal membrane-associated | cell-surface |
| | | | | | | protein | |
| mCG2748 | Cd24a | CD24a antigen | | | CD24 | CD24 antigen (small cell lung | cell-surface |
| | | | | | | carcinoma cluster 4 antigen) | |
| mCG1269 | Edg3 | Endothelial differentiation, | | | EDG3 | Endothelial differentiation, | cell-surface |
| | | sphingolipid G-protein- | | | | sphingolipid G-protein- | |
| | | coupled receptor 8 | | | | coupled receptor 3 | |
| mCG16856 | Itgp | Integrin-associated protein | | | CD47 | CD47 antigen (Rh-related | cell-surface |
| | | | | | | antigen, integrin-associated | |
| | | | | | | signal transducer) | |
| mCG51514 | Kcnj16 | Potassium inwardly-rectifying | | | KCNJ16 | Potassium inwardly-rectifying | cell-surface |
| | | channel, subfamily J, member | | | | channel, subfamily J, member | |
| | | 16 | | | | 16 | |
| mCG5936 | Kcnk5 | Potassium channel, subfamily | | | KCNK5 | Potassium channel, subfamily | cell-surface |
| | | K, member s | | | | K, member 5 (TASK-2) | |
| mCG22845 | Kcnn4 | Potassium intermediate/small | | | KCNN4 | Potassium intermediate/small | cell-surface |
| | | conductance calcium- | | | | conductance calcium- | |
| | | activated channel, subfamily | | | | activated channel, subfamily | |
| | | | | | | N, member 4 | |
| mCG15918 | Ltb | Lymphotoxin B | | | LTB | Lymphotoxin beta (TNF | cell-surface |
| | | | | | | superfamily, member 3) | |
| mCG4493 | Ly108 | Lymphocyte antigen 108 | | | Unknown | Unknown | cell-surface |
| mCG2784 | Ly6i | Lymphocyte antigen 6 | | | Human | Human homolog of Ly6i | cell-surface |
| | | complex, locus I | | | homolog of | | |
| | | | | | Ly6i | | |
| mCG23500 | mouse homologue | Mouse homolog of EMILIN | | | EMILIN | Elastin microfibril interfac | cell-surface |
| | of EMILIN | | | | | located protein | |
| mCG14198 | Mrc1 | Mannose receptor, C type 1 | | | MRC1 | Mannose receptor, C type 1 | cell-surface |
| mCG5780 | Ninj2 | Ninjurin 2 | | | NINJ2 | Ninjurin 2 | cell-surface |
| mCG22798 | Nphs1 | Nephrosis 1 homolog, nephrin | | | NPHS1 | Nephrosis 1, congenital, | cell-surface |
| | | (human) | | | | Finnish type (nephrin) | |
| mCG19462 | Sema4b | Sema domain, | | | SEM4B | Sema domain, | cell-surface |
| | | immunoglobulin domain (Ig), | | | | immunoglobulin domain (Ig), | |
| | | transmembrane domain ™ | | | | transmembrande domain | |
| | | and short cytoplamic domain, | | | | (TM) and short cytoplasmic | |
| | | (semaphoring) 4B | | | | domain, (semaphoring) 4B | |
| mCG11191 | Tm9sf2 | Transmembrane 9 | | | TM95F2 | Transmembrane 9 | cell-surface |
| | | superfamily member 2 | | | | superfamily member 2 | |
| mCG6955 | Tnfrsf17 | Tumor necrosis factor | | | TNFRSF17 | Tumor necrosis factor | cell-surface |
| | | receptor superfamily, member | | | | receptor superfamily, member | |
| | | 17 | | | | 17 | |
| mCG8017 | Apobec2 | Apolipoprotein B editing | | | APOBEC2 | Apolipoprotein B mRNA | enzyme |
| | | complex 2 | | | | editing enzyme, catalytic | |
| | | | | | | polypeptide-like 2 | |
| mCG3809 | Btd | Biotinidase | | | BTD | Biotinidase | enzyme |
| mCG15177 | Cds2 | CDP-diacylglycerol synthase | | | CD52 | CDP-diacylglycerol synthase | enzyme |
| | | (phosphatidate | | | | (phosphatidate | |
| | | cytidylyltransferase) 2 | | | | cytidylyltransferase) 2 | |
| mCG16418 | Clpx | Caseinolytic protease X | | | CLPX | ClpX caseinolytic protease x | enzyme |
| | | (E. coli) | | | | homolog (E. coli) | |
| mCG50857 | Ddx19 | DEAD/H (Asp-Glu-Ala- | | | DDX19 | DEAD/H (Asp-Glu-Ala- | enzyme |
| | | Asp/His) box polypeptide 19 | | | | Asp/His) box polypeptide 19 | |
| | | | | | | (DBPS homolog, yeast) | |
| mCG11315 | Ddx21 | DEAD/H (Asp-Glu-Ala- | | | DDX21 | DEAD/H (Asp-Glu-Ala- | enzyme |
| | | Asp/His) box polypeptide 21 | | | | Asp/His) box polypeptide 21 | |
| | | (RNA helicase II/GU) | | | | | |
| mCG15707 | Dnmt2 | DNA methyltransferase 2 | | | DNMT2 | DNA (cytosine-5-)- | enzyme |
| | | | | | | methyltransferase 2 | |
| mCG14354 | Dqx1 | DEAQ RNA-dependent | | | DQX1 | DEAQ RNA-dependent | enzyme |
| | | Atpase | | | | Atpase DQX1 | |
| mCG8426 | Hdac7a | Histone deacetylase 7A | | | HDAC7A | Histone deacetylase 7A | enzyme |
| mCG5273 | Lce-pending | Long chain fatty acyl elongase | | | LCE | Long chain fatty acyl elongase | enzyme |
| mCG14414 | Mgat1 | Mannoside | | | MGATI | Mannosyl (alpha-1,3-)- | enzyme |
| | | acetylglucosaminyltransferase 1 | | | | glycoprotein beta-1,2-N- | |
| | | | | | | acetylglucosaminyltransferase | |
| mCG16439 | mouse homologue | Mouse homolog of CILP | | | CILP | Cartilage intermediate layer | enzyme |
| | of CILP | | | | | protein, nucleotide | |
| | | | | | | pyrophosphohydrolase | |
| mCG21395 | mouse homologue | Mouse homolog of NOH61 | | | NOH61 | Putative nucleolar RNA | enzyme |
| | of NOH61 | | | | | helicase | |
| mCG11229 | Nudel-pending | Nuclear distribution gene E- | | | NUDEL | LIS1-interacting protein | enzyme |
| | | like | | | | NUDEL; endooligopeptidase A | |
| mCG2309 | Pah | Phynylalaline hydroxylase | | | PAH | Phenylalaline hydroxylase | enzyme |
| mCG9046 | Pdi1 | Peptidyl arginie deiminase, | | | PADIL | Peptidyl arginie deiminase, | enzyme |
| | | type I | | | | type I | |
| mCG17125 | Ppia | Peptidylpropyl isomerase A | | | PPIA | Peptidylprolyl isomerase A | enzyme |
| | | | | | | (cyclophilin A) | |
| mCG19617 | Prps1 | Phosphoribosyl | | | PRPS1 | Phosphoribosyl | enzyme |
| | | pyrophosphate sythetase 1 | | | | pyrophosphate synthetase 2 | |
| mCG18746 | Ptgds | Prostaglandin D2 synthase | | | PTGDS | Prostaglandin D2 synthase | enzyme |
| | | (21 kDa, brain) | | | | (21 kD, brain) | |
| mCG15930 | Vars2 | Valyl-tRNA synthetase 2 | | | VAR2 | Valyl-tRNA synthetase 2 | enzyme |
| mCG13588 | Dagk4 | Diacylglycerol kinase, delta | | | DGKQ | Diacylglycerol kinase, theta | kinase |
| | | (110 kDa) | | | | (110 kD) | |
| mCG22407 | mouse homologue | Mouse homolog of PSK | | | PSK | Prostate derived STE20-like | kinase |
| | of PSK | | | | | kinase PSK | |
| mCG1461 | Nme2 | Expressed in non-metastatic | | | NME2 | Non-metastic cells 2, protein | kinase |
| | | cells 2, protein | | | | (NM2JB) expressed in | |
| | | (NM23B)nucleoside | | | | | |
| | | diphosphate kinase) | | | | | |
| mCG14256 | Snf1lk | SNF1-like kinase | | | SNF1LK | SNF1-like kinase | kinase |
| mCG17800 | Tyki | Thymidylate kinase family | | | Human | Human homolog of Tyki | kinase |
| | | LPS-inducible member | | | homolog of | | |
| | | | | | Tyki | | |
| mCG15978 | Dusp10 | Dual specificity phosphatase | | | DUSP10 | Dual specificity phosphatase | phosphatase |
| | | 10 | | | | 10 | |
| mCG13074 | Inpp4a | Inositol polyphoshate-4- | | | INPP4A | Inositol polyphosphate-4- | phosphatase |
| | | phosphatase, type I, 107 kD | | | | phosphatase, type I, 107 kD | |
| mCG17293 | Inpp5b | Inositol polyphosphate-5- | | | INPP5B | Inositol polyphosphate-5- | phosphatase |
| | | phosphatase, 75 kDa | | | | phosphatase, 75 kD | |
| mCG2895 | Ptpn5 | Protein tyrosine phosphatase, | | | PTPNS | Protein tyrosine phosphatase, | phosphatase |
| | | non-receptor type 5 | | | | non-receptor type 5 (striatum- | |
| | | | | | | enriched) | |
| mCG8259 | Il16 | Interleukin 16 | | | IL16 | Interleukin 16 (lymphocyte | secreted |
| | | | | | | chemoattractant factor) | factors |
| mCG11329 | Prg | Proteoglycan, secretory | | | PRG1 | Proteoglycan, secretory | secreted |
| | | granule | | | | granule | factors |
| mCG11627 | Scya4 | Small inducible cytokine A4 | | | SCYA4 | Small inducible cytokine A4 | secreted |
| | | | | | | | factors |
| mCG9005 | Akap7 | A kinase (PRKA) anchor | | | AKAP7 | A kinase (PRKA) anchor | signaling |
| | | protein 7 | | | | protein 7 | |
| mCG18038 | Api5 | Apoptosis inhibitory protein 5 | | | AP15 | Apoptosis inhibitory 5 | signaling |
| mCG23071 | Arfrp1 | ADP-ribosylation factor | | | ARFRP1 | ADP-ribosylation factor | signaling |
| | | related protein 1 | | | | related protein 1 | |
| mCG15346 | Arhgap14-pending | Rho GTPase activating | | | SRGAP3 | SLIT-ROBO Rho GTPase- | signaling |
| | | protein 14 | | | | activating protein 3 | |
| mCG2796 | Cish2 | Cytokine inducible SH2- | | | STATI2 | STAT induced STAT | signaling |
| | | containing protein 2 | | | | inhibitor-2 | |
| mCG4112 | DappI | Dual adaptor for | | | DAPP1 | Dual adaptor of | signaling |
| | | phosphotyrosine and J- | | | | phosphotyrosine and 3- | |
| | | phophoinositidas 1 | | | | phosphoinositides | |
| mCG21802 | Fabp6 | Fatty acid binding protein 6, | | | FABP6 | Fatty acid binding protein 6, | signaling |
| | | ileal (gastrotropin) | | | | ileal (gastrotropin) | |
| mCG23117 | Fkbp8 | FK506 binding protein 8 (38 kDa) | | | FKBP8 | FK506 binding protein 8 (38 kD) | signaling |
| mCG20993 | Flizl-pending | Fatal liver zinc finger 1 | | | Human | Human homolog of Flizl- | signaling |
| | | | | | homolog of | pending | |
| | | | | | Flizl-pen | | |
| mCG1442 | Hint | Histidine triad nucleotide | | | HINT | Histidine triad nucleotide | signaling |
| | | binding protein | | | | binding protein 1 | |
| mCG8214 | Ier5 | Immediate early response 5 | | | IERS | Immediate early response 5 | signaling |
| mCG5743 | Jundp2-pending | Jun dimerization protein 2 | | | Unknown | Unknown | signaling |
| mCG3955 | Lmo6 | LIM only 6 | | | LMO6 | LIM domain only 6 | signaling |
| mCG50212 | MidI | Midline 1 | | | MIDI | Midline 1 (Opitz/BBB | signaling |
| | | | | | | syndrome) | |
| mCG15699 | mouse homologue | Mouse homolog of AKAP13 | | | AKAP13 | A kinase (PRKA) anchor | signaling |
| | of AKAP13 | | | | | protein 13 | |
| mCG16853 | mouse homologue | Mouse homolog of BIN2 | | | BIN2 | Bridging integrator 2 | signaling |
| | of BIN2 | | | | | | |
| mCG16763 | mouse homologue | Mouse homolog of CEZANNE | | | CEZANNE | Cellular zinc finger anti-NF- | signaling |
| | of CEZANNE | | | | | kappaB Cezanne | |
| mCG19747 | mouse homologue | Mouse homolog of CHD2 | | | CHD2 | Chromodomain helicase DNA | signaling |
| | of CHD2 | | | | | binding protein 2 | |
| mCG4278 | mouse homologue | Mouse homolog of MBLL | | | MBLL | C3H-type zinc finder protein; | signaling |
| | of MBLL | | | | | similar to D. melanogaster | |
| | | | | | | muschleblind B | |
| mCG1408 | mouse homologue | Mouse homolog of SLC16A10 | | | SLC16A10 | Solute carrier family 16 | signaling |
| | of SLC16AI0 | | | | | (monocarboxylic acid | |
| | | | | | | transporters), member 10 | |
| mCG19635 | mouse homologue | Mouse homolog of SLC16A6 | | | SLC16A6 | Solute carrier family 16 | signaling |
| | of SLC16A6 | | | | | (monocarboxylic acid | |
| | | | | | | transporters), member 6 | |
| mCG15231 | mouse homologue | Mouse homolog of SLC17A5 | | | SLC17A5 | Solute carrier family 17 | signaling |
| | of SLC17A5 | | | | | (anion/sugar transporter), | |
| | | | | | | member 5 | |
| mCG1770 | mouse homologue | Mouse homolog of TAF5L | | | TAF5L | TAFS-like RNA polymerase | signaling |
| | of TAF5L | | | | | II, p300/CBP-associated factor | |
| | | | | | | (PCAF)-associated factor, 65 KD | |
| mCG17135 | mouse homologue | Mouse homolog of | | | U1SNRNPBP | U1-snRNP binding protein | signaling |
| | of U1SNRNPBP | U1SNRNPBP | | | | homolog (70 kD) | |
| mCG21531 | mouse homologue | Mouse homolog of ZNF8 | | | ZNF3 | Zinc finger protein 8 (clone | signaling |
| | of ZNF8 | | | | | HF.18) | |
| mCG2820 | Mtap7 | Microtubule-associated | | | MAP7 | Microtubule-associated | signaling |
| | | protein 7 | | | | protein 7 | |
| mCG10776 | Myo1c | Myosin Ic | | | MYO1C | Myosin IC | signaling |
| mCG18907 | Nkx2-3 | NK2 transcription factor | | | LOC159296 | Similar to HOMEO protein | signaling |
| | | related, locus 3 (Drosophila) | | | | nkX-2.3 | |
| mCG19161 | Nsf | N-ethylmaleimide sensitve | | | NSF | N-ethylmaleimide-sensitive | signaling |
| | | fusion protein | | | | factor | |
| mCG51109 | Pcdh9 | Protocadherin 9 | | | PCDH9 | Protocadherin 9 | signaling |
| mCG5444 | Pkig | Protein kinase inhibitor, | | | PKIG | Protein kinase (cAMP- | signaling |
| | | gamma | | | | dependent, catalyctic) | |
| | | | | | | inhibitor gamma | |
| mCG5911 | Prdx2 | Peroxiredoxin 2 | | | PRDX2 | Peroxiredoxin 2 | signaling |
| mCG13896 | Pscd1 | Pleckstrin homology, sec7 and | | | PSCD1 | Pleckstrin homology, sec7 and | signaling |
| | | coiled/coil domains 1 | | | | coiled/coil domains 1 | |
| | | | | | | (cytohesin 1) | |
| mCG4504 | Psmb1 | Proteasome (prosome, | | | PSMB1 | Proteasome (prosome, | signaling |
| | | macropain) subunit, beta type 1 | | | | macropain) subunit, beta type 1 | |
| mCG22049 | Psme1 | Proteasome (prosome, | | | PSME1 | Proteasome (prosome, | signaling |
| | | macropain) 28 subunit, alpha | | | | macropain) activator subunit | |
| | | | | | | 1 (PA28 alpha) | |
| mCG220-48 | Psme2 | Proteasome (prosome, | | | PSME2 | Proteasome (prosome, | signaling |
| | | macropain)28 subunit, beta | | | | macropain) activator subunit | |
| | | | | | | 2 (PA 28 beta) | |
| mCG14500 | Rgl1 | Ral guanine nucleotide | | | RGL | RalGDS-like gene | signaling |
| | | dissociation stimulator, -like 1 | | | | | |
| mCG13780 | Ril-pending | Reversion induced TIM gene | | | RIL | LTM domain protein | signaling |
| mCG15821 | Sax1 | Spinal cord axial homeobox | | | SAX1 | Spastic ataxia 1 (dominant) | signaling |
| | | gene 1 | | | | | |
| mCG15477 | Slc14a2 | Solute carrier family 14 (urea | | | SLC14A2 | Solute carrier family 14 (urea | signaling |
| | | transporter), member 2 | | | | transporter), member 2 | |
| mCG12717 | Slc7a1 | Solute carrier family 7 | | | SLC7A1 | Solute carrier family 7 | signaling |
| | | (cationic amino acid | | | | (cationic amino acid | |
| | | transporter, y⁺ system) | | | | transporter, y⁺ system) | |
| | | member 1 | | | | member 1 | |
| mCG20789 | Slc7a11 | Solute family 7 (cationic | | | SLC7A11 | Solute family 7 (cationic | signaling |
| | | amino acid transporter, y⁺ | | | | amino acid transporter, y⁺ | |
| | | system) member 11 | | | | system) member 11 | |
| mCG6705 | Swap70 | SWAP complex protein, 70 kDa | | | SWAP70 | SWAP-70 protein | signaling |
| mCG14853 | Txnip | Thioredoxin interacting | | | TXNIP | Thioredoxin interacting | signaling |
| | | protein | | | | protein | |
| mCG12718 | Ub13 | Ubiquitin-like 3 | | | UBLJ | Ubiquitin-like 3 | signaling |
| mCG18751 | Clic3 | Chloride intracellular channel 3 | | | CLIC3 | Chloride intracellular channel 3 | structure |
| mCG13494 | Gtl1-13 | Gene trap locus 1-13 | | | NUP160 | Nucleoporin 160 kD | structure |
| mCG19857 | mouse homologue | Mouse homolog of NOL5A | | | NOL5A | Nucleolar protein 5A (56 kD | structure |
| | of NOL5A | | | | | with KKE/D repeat) | |
| mCG7855 | Vdac2 | Voltage-dependent anion | | | VDAC2 | Voltage-dependent anion | structure |
| | | channel 2 | | | | channel 2 | |
| mCG7737 | ABT1-pending | Activator of basal | | | ABT1 | TATA-binding protein- | transcription |
| | | transcription | | | | binding protein | |
| mCG2534 | Ctbp1 | C-termical binding protein 1 | | | CTBP1 | C-terminal binding protein 1 | transcription |
| mCG20120 | Dermo1 | Dermis expressed 1 | | | DERMO1 | Dermis expressed 1 | transcription |
| mCG20096 | Ebf | Early B-cell factor | | | EBF | Early B-cell factor | transcription |
| mCG5050 | Elf4 | E74-like factor 4 (ets domain | | | ELF4 | E74-like factor 4 (ets domein | transcription |
| | | transcription factor) | | | | transcription factor) | |
| mCG10284 | Ldb1 | LIM domain binding 1 | | | LDB1 | LIM domain binding 1 | transcription |
| mCG15360 | mouse homologue | Mouse homolog of NR1D1 | | | NR1D1 | Nuclear receptor subfamily 1, | transcription |
| | of NR1D1 | | | | | group D, member 1 | |
| mCG8451 | mouse homologue | Mouse homolog of ZER6 | | | ZER6 | Zinc finger DNA binding | transcription |
| | of ZER6 | | | | | protein ZER6 | |
| mCG15860 | Rest | RE1-silencing transcription | | | REST | RE1-silencing transcription | transcription |
| | | factor | | | | factor | |
| mCG4503 | Tbp | TATA box binding protein | | | TBP | TATA box binding protein | transcription |
| mCG14947 | Zfp238 | zinc finger protein 238 | | | ZNF238 | zinc finger protein 238 | transcription |
| mCG23383 | Zfp287 | zinc finger protein 287 | | | ZNF287 | zinc finger protein 287 | transcription |
| mCG12285 | Zfp319 | zinc finger protein 319 | | | KIAA1388 | KIAA1388 | transcription |
| mCG15799 | Lrrc2 | Laucine-rich repeat- | | | KRRC2 | Laucine-rich repeat- | unknown |
| | | containing 2 | | | | containing 2 | |
| mCG18182 | Satb1 | Special AT-rich sequence | | | SATB1 | Special AT-rich sequence | unknown |
| | | binding protein 1 | | | | binding protein 1 (binds to | |
| | | | | | | nuclear matriz/scaffold | |
| mCG53918 | Slfn4 | Schlafen 4 | | | unknown | | unknown |
| mCG10290 | unknown | | unknown | AC108484 Mus musculus | unknown | unknown | unknown |
| | | | | clone ct7-293m19 map 19 | | | |
| | | | | strain 129/Sv, 170321 bp | | | |
| | | | | genomic DNA | | | |
| mCG10613 | unknown | | NM_172928 | | unknown | unknown | unknown |
| | | | Human: KIAA1765 protein, | | | | |
| | | | AB051552, myosin light chain | | | | |
| | | | kinase | | | | |
| mCG11234 | unknown | | unknown | NM_177060 | unknown | unknown | unknown |
| mCG11325 | unknown | | unknown | AK003009, AK035659 | unknown | unknown | unknown |
| mCG11355 | unknown | | NM_138665 Human: | | unknown | unknown | unknown |
| | | | GENBANK: NM_007101 | | | | |
| | | | sarcosine dehydrogenase | | | | |
| | | | (SARDH) | | | | |
| mCG11803 | unknown | | BC021749, synovial sarcoma, | | unknown | unknown | unknown |
| | | | X breakpoint 2 interacting | | | | |
| | | | protein, AB023140, | | | | |
| | | | KIAA0923 | | | | |
| mCG11817 | unknown | | BC021749, synovial sarcoma, | | unknown | unknown | unknown |
| | | | X breakpoint 2 interacting | | | | |
| | | | protein, AB023140, | | | | |
| | | | KIAA0923 | | | | |
| mCG12566 | unknown | | NM_172688, MAP3K, | | unknown | unknown | unknown |
| | | | transforming growth factor- | | | | |
| | | | beta-activated kinase 1 | | | | |
| mCG12630 | unknown | | BC037187, MICROSOMAL | | unknown | unknown | unknown |
| | | | SIGNAL PEPTIDASE S26- | | | | |
| | | | RELATED | | | | |
| mCG12824 | unknown | | NM_013931 Mus musculus | | unknown | unknown | unknown |
| | | | mitogen-activated protein | | | | |
| | | | kinase 8 interacting protein | | | | |
| | | | 3 (Mapk8ip3) | | | | |
| mCG13346 | unknown | | unknown | BC025489 | unknown | unknown | unknown |
| mCG14143 | unknown | | unknown | AK004855 | unknown | unknown | unknown |
| mCG14155 | unknown | | AB006141, AK055716, | | unknown | unknown | unknown |
| | | | AK001225, IGFBP-like | | | | |
| | | | protein | | | | |
| mCG14342 | unknown | | mhook homolog 2 | | unknown | unknown | unknown |
| | | | (Drosophila), BC002226, | | | | |
| | | | BC026609, BC019486, | | | | |
| | | | AF044924, BC012443, | | | | |
| | | | HOOK2 | | | | |
| mCG15141 | unknown | | XP_130301 AF086541, | | unknown | unknown | unknown |
| | | | DEATH-ASSOCIATED | | | | |
| | | | PROTEIN 1 (DAP-1) | | | | |
| mCG15321 | unknown | | NM_025914, ARP6 actin- | | unknown | unknown | unknown |
| | | | related protein 6 homolog | | | | |
| | | | (yeast) NM_022496 actin- | | | | |
| | | | related protein 6 (ACTR6) | | | | |
| mCG16761 | unknown | | cisplatin resistance | | Cra | unknown | unknown |
| | | | associated alpha protein, | | | | |
| | | | NM_006697 | | | | |
| mCG16858 | unknown | | huntingtin-interacting | | unknown | unknown | unknown |
| | | | protein-1 protein interactor, | | | | |
| | | | NM_028680 Estrogen-related | | | | |
| | | | receptor beta like 1 (Esrrbl1), | | | | |
| | | | NM_018010 | | | | |
| mCG17127 | unknown | | unknown | NP_067405, AK096697.1 | unknown | unknown | unknown |
| mCG17140 | unknown | | unknown | BC003324, NP_659495 | unknown | unknown | unknown |
| mCG17142 | unknown | | XP_227169 similar to | | unknown | unknown | unknown |
| | | | Histone-lysine N- | | | | |
| | | | methyltransferase, H4 lysine- | | | | |
| | | | 20 specific (Histone H4-K20 | | | | |
| | | | methyltransferase) (H4-K20- | | | | |
| | | | HMTase) (SET domain- | | | | |
| | | | containing protein 8) | | | | |
| | | | (PR/SET domain-containing | | | | |
| | | | protein 07) (PE/SET07) (PR- | | | | |
| | | | Set7) [Rattus norvegicus]; | | | | |
| | | | SET domain-containing | | | | |
| | | | protein 8, NM_020382, | | | | |
| | | | PR/SET domain containing | | | | |
| | | | protein 07 (SET07) | | | | |
| mCG17547 | unknown | | unknown | NP_079334, NP_0793341, | unknown | unknown | unknown |
| | | | | hypothetical protein | | | |
| | | | | FLJ23229 tripartite motif- | | | |
| | | | | containing 46 (TRIM46), | | | |
| | | | | NM_025058.1 | | | |
| mCG17569 | unknown | | DPM3 dolichyl-phosphate | | unknown | unknown | unknown |
| | | | mannosyltransferase | | | | |
| | | | polypeptide 3 isoform 1; | | | | |
| | | | prostin 1; dolichol-phosphate | | | | |
| | | | mannose synthase subunit 3; | | | | |
| | | | dolichyl-phosphate beta-D- | | | | |
| | | | mannosyltransferase subunit | | | | |
| | | | 3; mannose-P- | | | | |
| | | | dolicholsynthase subunit 3, | | | | |
| | | | NP_061846.2, NM_018973.3 | | | | |
| mCG17751 | unknown | | lung-inducible neuralized- | | unknown | unknown | unknown |
| | | | related C3HC4 RING domain | | | | |
| | | | protein, XP_129868 | | | | |
| | | | NM_138397.1, hypothetical | | | | |
| | | | protein BC012317 | | | | |
| | | | (LOC93082) | | | | |
| mCG17799 | unknown | | VHSV-induced-like protein, | | unknown | unknown | unknown |
| | | | AAF60314, vipirin (cig5); | | | | |
| | | | similar to inflammatory | | | | |
| | | | response protein 6, | | | | |
| | | | NP_542388, NM_080657 | | | | |
| mCG17802 | unknown | | XP_234017.1\| similar to | | unknown | unknown | unknown |
| | | | thymidylate kinase family | | | | |
| | | | LPS-inducible member; | | | | |
| | | | thymidylate kinase | | | | |
| | | | homologue BC016969 | | | | |
| mCG17918 | unknown | | unknown | XP_166091.2, KIAA1224 | unknown | unknown | unknown |
| mCG18034 | unknown | | unknown | XP_130328.3\| RIKEN | unknown | unknown | unknown |
| | | | | cDNA 9130020K17, | | | |
| | | | | gb\|AAH33000.1\| | | | |
| | | | | hypothetical protein | | | |
| | | | | FLJ10890, NM_018259 | | | |
| mCG1850 | unknown | | unknown | NP_598552.1\| hypothetical | unknown | unknown | unknown |
| | | | | protein D8Ertd594e; | | | |
| | | | | NM_024949, BH3-only | | | |
| | | | | member B protein (BOMB) | | | |
| mCG18663 | unknown | | ref\|NP_620706.1\| SWI/SNF | | unknown | unknown | unknown |
| | | | related matrix-associated | | | | |
| | | | actin-dependent regulator of | | | | |
| | | | chromatin c2 isoform b; | | | | |
| | | | mammalian chromatin | | | | |
| | | | □emodelling complex BRG1- | | | | |
| | | | associated factor 170; | | | | |
| | | | chromatin □emodelling | | | | |
| | | | complex BAF170 subunit; | | | | |
| | | | SWI3-like protein; SWI/SNF | | | | |
| | | | complex 170 kDa subunit | | | | |
| | | | [Homo sapiens] | | | | |
| mCG18737 | unknown | | unknown | NP_705785.1\| hypothetical | unknown | unknown | unknown |
| | | | | protein MGC36831 | | | |
| | | | | AAH02613 Unknown | | | |
| | | | | (protein for | | | |
| | | | | IMAGE: 3161564) | | | |
| mCG20276 | unknown | | unknown | 1110019I14Rik, | unknown | unknown | unknown |
| | | | | AAF68024.1\|AF236061_1 | | | |
| | | | | RING-finger binding protein | | | |
| | | | | [Oryctolagus cuniculus] | | | |
| mCG20905 | unknown | | unknown | Similar to hypothetical | unknown | unknown | unknown |
| | | | | protein FLJ10385 | | | |
| | | | | gb\|AAH02336.1\| AAH02336 | | | |
| | | | | hypothetical protein | | | |
| | | | | FLJ10885 [Homo sapiens], | | | |
| | | | | NM_018081 | | | |
| mCG20994 | unknown | | unknown | ref\|XP_203881.1\| RIKEN | unknown | unknown | unknown |
| | | | | cDNA 4631426G04 | | | |
| mCG21403 | unknown | | unknown | glycoprotein 25L related | unknown | unknown | unknown |
| mCG21505 | unknown | | unknown | MNCb-2717 NM_153357.1, | unknown | unknown | unknown |
| | | | | FLJ90193 | | | |
| mCG21529 | unknown | | ref\|NP_631888.1\| pigpen | | unknown | unknown | unknown |
| | | | [Mus musculus] | | | | |
| | | | gb\|AAH26062.1\|, | | | | |
| | | | NM_004960, Homo sapiens | | | | |
| | | | fusion, derived from t(12; 16) | | | | |
| | | | malignant liposarcoma | | | | |
| | | | (FUS), mRNA | | | | |
| mCG21530 | unknown | | unknown | ref\|XP_146056.1\| similar | unknown | unknown | unknown |
| | | | | to zinc finger protein 111 | | | |
| | | | | [Mus musculus] | | | |
| | | | | ref\|NP_078982.2\| | | | |
| | | | | hypothetical protein | | | |
| | | | | FLJ13479 [Homo sapiens], | | | |
| | | | | NM_024706 | | | |
| mCG21803 | unknown | | FABP6 GASTROTROPIN | | unknown | unknown | unknown |
| | | | NM_008375 | | | | |
| mCG22014 | unknown | | unknown | ref\|XP_150117.1\| RIKEN | unknown | unknown | unknown |
| | | | | cDNA 2610044O15 | | | |
| mCG22045 | unknown | | unknown | gb\|AAH37001.1\| Unknown | unknown | unknown | unknown |
| | | | | (protein for MGC: 46990) | | | |
| | | | | [Mus musculus]( | | | |
| mCG22386 | unknown | | unknown | gb\|AAH23442.1\| RIKEN | unknown | unknown | unknown |
| | | | | cDNA 111003ZO16 gene | | | |
| | | | | ref\|XP_058805.1\| | | | |
| | | | | hypothetical protein | | | |
| | | | | XP_058805 | | | |
| mCG2258 | unknown | | ref\|XP_141255.2\| similar to | | unknown | unknown | unknown |
| | | | tetraspanin similiar to | | | | |
| | | | uroplakin 1 [Homo sapiens] | | | | |
| | | | [Mus musculus] | | | | |
| | | | ref\|NP_570189.1\| | | | | |
| | | | tetraspanin similar to | | | | |
| | | | uroplakin 1 [Homo sapiens], | | | | |
| | | | NM_130783.1 | | | | |
| mCG22772 | unknown | | unknown | ref\|XP_133287.2\| RIKEN | unknown | unknown | unknown |
| | | | | cDNA 2310022A10 | | | |
| mCG23032 | unknown | | ref\|XP_138472.1\| similar to | | unknown | unknown | unknown |
| | | | olfactory receptor MOR256-10 | | | | |
| | | | Homo sapiens olfactory | | | | |
| | | | receptor, family 2, subfamily | | | | |
| | | | B, member 2 (OR2B2), | | | | |
| | | | NM_033057 | | | | |
| mCG23035 | unknown | | gb\|AAH19757.2\| Similar to | | unknown | unknown | unknown |
| | | | germinal histone H4 gene | | | | |
| mCG23069 | unknown | | unknown | XM_194070 Mus | unknown | unknown | unknown |
| | | | | musculus hypothetical | | | |
| | | | | protein LOC269400 | | | |
| mCG23075 | unknown | | AJ413952, Mus musculus | | unknown | unknown | unknown |
| | | | Arfrp1 gene for ARF-related | | | | |
| | | | protein 1 | | | | |
| mCG23120 | unknown | | FKDP8 and ELL NM_010223 | | unknown | unknown | unknown |
| mCG2543 | unknown | | unknown | ref\|XP_132022.1\| RIKEN | unknown | unknown | unknown |
| | | | | cDNA 2610033H07 | | | |
| mCG2824 | unknown | | unknown | gb\|AAM94175.1\| Ahi-1 | unknown | unknown | unknown |
| | | | | isoform I [Mus musculus] | | | |
| | | | | Homo sapiens hypothetical | | | |
| | | | | protein FLJ20069, | | | |
| | | | | NM_017651 | | | |
| mCG2947 | unknown | | unknown | ref\|XP_134875.3\| RIKEN | unknown | unknown | unknown |
| | | | | cDNA 2310020N23 | | | |
| | | | | (subfamily: myosin-I) | | | |
| mCG3038 | unknown | | unknown | ref\|XP_127418.2\| | unknown | unknown | unknown |
| | | | | expressed sequence | | | |
| | | | | AI843389 | | | |
| | | | | ref\|NP_699185.1\| | | | |
| | | | | hypothetical protein, | | | |
| | | | | NM_153354 | | | |
| mCG3729 | unknown | | unknown | ref\|XP_130718.1\| similar | unknown | unknown | unknown |
| | | | | to homo sapiens | | | |
| | | | | chromosome 20 open | | | |
| | | | | reading frame 98; similar to | | | |
| | | | | mouse VMP Homo sapiens | | | |
| | | | | chromosome 20 open | | | |
| | | | | reading frame 98 | | | |
| | | | | (C20orf98), NM_024958 | | | |
| mCG3760 | unknown | | unknown | gb\|AAH11270.1\| Similar to | unknown | unknown | unknown |
| | | | | DEAD/H (Asp-Glu-Ala- | | | |
| | | | | Asp/His) box polypeptide 19 | | | |
| | | | | Homo sapiens hypothetical | | | |
| | | | | protein FLJ11126 | | | |
| | | | | (FLJ11126), NM_018332 | | | |
| mCG50409 | unknown | | unknown | gb\|AAH37188.1\| Unknown | unknown | unknown | unknown |
| | | | | (protein for | | | |
| | | | | IMAGE: 3663008), | | | |
| | | | | ref\|XP_090783.2\| similar | | | |
| | | | | to RIKEN cDNA | | | |
| | | | | 1110028A07 RIKEN cDNA | | | |
| | | | | 1110028A07 | | | |
| mCG50651 | unknown | | unknown | | unknown | unknown | unknown |
| mCG5068 | unknown | | unknown | Knowles Solter mouse 2 cell | unknown | unknown | unknown |
| | | | | Mus musculus, AI503444 | | | |
| mCG5070 | unknown | | unknown | gb\|AAH26447.1\| RIKEN | unknown | unknown | unknown |
| | | | | cDNA 9030425E11 | | | |
| | | | | ref\|NP_079045.1\| | | | |
| | | | | hypothetical protein | | | |
| | | | | FLJ22415, NM_024769 | | | |
| mCG51393 | unknown | | unknown | AL591208 Mouse DNA | unknown | unknown | unknown |
| | | | | sequence from clone RP23- | | | |
| | | | | 149I2 on chromosome 12 | | | |
| mCG52252 | unknown | | unknown | gb\|AAH25220.1\| | unknown | unknown | unknown |
| | | | | hypothetical protein | | | |
| | | | | E030024M05, NM_172574 | | | |
| mCG52498 | unknown | | ref\|XP_127147.1\| RIKEN | | unknown | unknown | unknown |
| | | | cDNA 2410018C03 CDCA4 | | | | |
| | | | gb\|AAK31075.1\|AF322239_1 | | | | |
| | | | hematopoietic progenitor | | | | |
| | | | protein, NM_017955 | | | | |
| mCG53009 | unknown | | unknown | ref\|XP_150419.1\| | unknown | unknown | unknown |
| | | | | hypothetical protein | | | |
| | | | | XP_150419 | | | |
| mCG53724 | unknown | | unknown | | unknown | unknown | unknown |
| mCG55023 | unknown | | unknown | ref\|XP_289994.1\| similar | unknown | unknown | unknown |
| | | | | to 1c7 protein | | | |
| mCG55075 | unknown | | PEPTIDYL-PROLYL CIS- | | unknown | unknown | unknown |
| | | | TRANS ISOMERASE A (EC | | | | |
| | | | 5.2.1.8) (PPIASE) | | | | |
| | | | (ROTAMASE) | | | | |
| | | | (CYCLOPHILIN A) | | | | |
| | | | (CYCLOSPORIN A- | | | | |
| | | | BINDING PROTEIN) (SP18). | | | | |
| | | | NM_008907 | | | | |
| mCG55198 | unknown | | unknown | AL844839 Mouse DNA | unknown | unknown | unknown |
| | | | | sequence from clone RP23- | | | |
| | | | | 191F2 on chromosome 2 | | | |
| mCG55265 | unknown | | ref\|XP_135750.1\| similar to | | unknown | unknown | unknown |
| | | | putative integral membrane | | | | |
| | | | transporter; lysosomal- | | | | |
| | | | associated transmembrane | | | | |
| | | | protein 4 beta [Homo | | | | |
| | | | sapiens] [Mus musculus] | | | | |
| mCG55512 | unknown | | unknown | AL590626 Mouse DNA | unknown | unknown | unknown |
| | | | | sequence from clone RP23- | | | |
| | | | | 143O13 on chromosome 13 | | | |
| mCG56069 | unknown | | unknown | | unknown | unknown | unknown |
| mCG56089 | unknown | | Novel gene; Srp9; Nm-012058 | | unknown | unknown | unknown |
| mCG56746 | unknown | | unknown | | unknown | unknown | unknown |
| mCG57132 | unknown | | kinesislike protein Kif-3 Nm- | | unknown | unknown | unknown |
| | | | 008444 | | | | |
| mCG57265 | unknown | | unknown | | unknown | unknown | unknown |
| mCG57617 | unknown | | Hu C/EBP induced protein | | unknown | unknown | unknown |
| | | | NM_030802 | | | | |
| mCG57827 | unknown | | SRIK1 glutamate receptor 5 | | unknown | unknown | unknown |
| | | | NM_175611 | | | | |
| mCG58254 | unknown | | ref\|XP_219735.1\| similar to | | unknown | unknown | unknown |
| | | | paraneoplastic antigen like 5; | | | | |
| | | | KIAA1934 protein; | | | | |
| | | | paraneoplastic antigen family | | | | |
| | | | 5 [Homo sapiens] [Rattus | | | | |
| | | | norvegicus] Homo sapiens | | | | |
| | | | paraneoplastic antigen like 5 | | | | |
| | | | (PNMA5), NM_052926 | | | | |
| mCG58345 | unknown | | NM-010512, IGF1 like | | unknown | unknown | unknown |
| | | | precursor | | | | |
| mCG5900 | unknown | | unknown | | unknown | unknown | unknown |
| mCG5905 | unknown | | TWIST2/Dermol NM_007855 | | unknown | unknown | unknown |
| mCG59368 | unknown | | unknown | ref\|XP_206491.1\| | unknown | unknown | unknown |
| | | | | hypothetical protein | | | |
| | | | | XP_206491 | | | |
| mCG59375 | unknown | | unknown | ref\|XP_140191.1\| | unknown | unknown | unknown |
| | | | | hypothetical protein | | | |
| | | | | XP_140191 | | | |
| mCG59533 | unknown | | unknown | AK052054 Mus musculus 12 | unknown | unknown | unknown |
| | | | | days embryo eyeball cDNA | | | |
| mCG59662 | unknown | | unknown | BC037471 Mus musculus | unknown | unknown | unknown |
| | | | | RIKEN cDNA 1110018I21 | | | |
| | | | | gene | | | |
| mCG59810 | unknown | | unknown | AL845543 Mouse DNA | unknown | unknown | unknown |
| | | | | sequence from clone RP23- | | | |
| | | | | 257O6 on chromosome 2 | | | |
| mCG59997 | unknown | | unknown | | unknown | unknown | unknown |
| mCG60526 | unknown | | actin-related protein 6 | | unknown | unknown | unknown |
| | | | (HARP6) NM_025914 | | | | |
| mCG60833 | unknown | | unknown | AL929552 Mouse DNA | unknown | unknown | unknown |
| | | | | sequence from clone RP23- | | | |
| | | | | 207J12 on chromosome 11 | | | |
| mCG61221 | unknown | | BRAIN-SPECIFIC | | unknown | unknown | unknown |
| | | | ANGIOGENESIS | | | | |
| | | | INHIBITOR 1-ASSOCIATED | | | | |
| | | | PROTEIN 2. [Source: RefSeq | | | | |
| | | | (NM_130862)] | | | | |
| mCG61661 | unknown | | unknown | | unknown | unknown | unknown |
| mCG61897 | unknown | | unknown | | unknown | unknown | unknown |
| mCG61907 | unknown | | unknown | | unknown | unknown | unknown |
| mCG61943 | unknown | | unknown | | unknown | unknown | unknown |
| mCG62177 | unknown | | unknown | mouse: novel cDNA; | unknown | unknown | unknown |
| | | | | 842043k14Riken | | | |
| mCG62971 | unknown | | ZINC FINGER HOMEOBOX | | unknown | unknown | unknown |
| | | | PROTEIN 1B (SMAD | | | | |
| | | | INTERACTING PROTEIN | | | | |
| | | | 1). NM015753 | | | | |
| mCG63537 | unknown | | vivininlike protein-VIS3; NM- | | unknown | unknown | unknown |
| | | | 0166 | | | | |
| mCG63601 | unknown | | unknown | H3051A08_1putative | unknown | unknown | unknown |
| | | | | protein in AL928567 Mouse | | | |
| | | | | DNA sequence from clone | | | |
| | | | | RP23-296K20 | | | |
| mCG64346 | unknown | | unknown | | unknown | unknown | unknown |
| mCG64382 | unknown | | unknown | AL935060 Mouse DNA | unknown | unknown | unknown |
| | | | | sequence from clone RP24- | | | |
| | | | | 151H6 on chromosome 2 | | | |
| mCG64398 | unknown | | Waspip; WASP | | unknown | unknown | unknown |
| | | | INTERACTING PROTEIN. | | | | |
| | | | Nm28-153138 | | | | |
| mCG65022 | unknown | | unknown | 2810036L13Riken | unknown | unknown | unknown |
| mCG65585 | unknown | | unknown | | unknown | unknown | unknown |
| mCG65785 | unknown | | Zfhx1b SMAD interacting | | unknown | unknown | unknown |
| | | | protein Q9ROG7 | | | | |
| mCG66128 | unknown | | unknown | mouse: BF019651 | unknown | unknown | unknown |
| mCG66379 | unknown | | unknown | NM_17692 Mus musculus | unknown | unknown | unknown |
| | | | | RIKEN cDNA A430057M04 | | | |
| | | | | gene (A430057M04Rik) | | | |
| mCG66776 | unknown | | unknown | AC087116 Mus Musculus | unknown | unknown | unknown |
| | | | | Strain C57BL6/J | | | |
| | | | | Chromosome 15˜RP23- | | | |
| | | | | 336B1 | | | |
| mCG66965 | unknown | | unknown | BB095650 RIKEN | unknown | unknown | unknown |
| mCG7831 | unknown | | unknown | ref\|NP_083617.1\| RIKEN | unknown | unknown | unknown |
| | | | | cDNA 1700028N11, | | | |
| | | | | NP_653248, NM_144647 | | | |
| mCG7856 | unknown | | unknown | ref\|XP_127600.1\| RIKEN | unknown | unknown | unknown |
| | | | | cDNA 1810030M08 | | | |
| mCG8424 | unknown | | unknown | ref\|XP_217033.1\| similar | unknown | unknown | unknown |
| | | | | to RIKEN cDNA | | | |
| | | | | 4930570C03 NP_060312 | | | |
| mCG9002 | unknown | | gb\|AAB61536.1\| alkaline | | unknown | unknown | unknown |
| | | | phosphodiesterase | | | | |
| | | | gb\|AAC51813.1\| | | | | |
| | | | phosphodiesterase | | | | |
| | | | I/nucleotide pyrophosphatase | | | | |
| | | | beta | | | | |
| mCG9537 | unknown | | ref\|NP_059052.1\| neural | | unknown | unknown | unknown |
| | | | visinin-like Ca2+-binding | | | | |
| | | | protein type 3 Mus musculus | | | | |
| | | | hippocalcin-like 1 (Hpcal1) | | | | |
| mCG9538 | unknown | | unknown | ref\|XP_126830.1\| RIKEN | unknown | unknown | unknown |
| | | | | cDNA 2410004P03 | | | |
| mCG9791 | unknown | | unknown | | unknown | unknown | unknown |
| mCG9792 | unknown | | unknown | AC109604 Mus musculus | unknown | unknown | unknown |
| | | | | clone rp22-298k22 strain | | | |
| | | | | 129Sv/CJ7 | | | |
| mCG9843 | unknown | | ref\|XP_232061.1\| similar to | | unknown | unknown | unknown |
| | | | B cell linker protein | | | | |
| mCG9875 | unknown | | unknown | AL845467 Mouse DNA | unknown | unknown | unknown |
| | | | | sequence from clone RP23- | | | |
| | | | | 435G20 on chromosome 2 | | | |
| mCG9877 | unknown | | gb\|AAC24206.1\| voltage | | unknown | unknown | unknown |
| | | | dependent calcium channel | | | | |
| | | | beta 4 subunit [Homo | | | | |
| | | | sapiens] | | | | |
| mCG9880 | unknown | | unknown | | unknown | unknown | unknown |

1-51. (canceled)

52. A method of preparing a polypeptide by expressing at least one murine genomic region involved in the development of cancer, said genomic region being selected from the group consisting of: Adam11, AI462175, Cd24a, Edg3, Itgp, Kcnj16, Kcnk5, Kcnn4, Ly108, Ly6i, mouse homologue of EMILIN, Mrc1, Ninj2, Nphs1, Sema4b, Tm9sf2, and Tnfrsf17, encoding cell surface proteins; Apobec2, Btd, Cds2, Clpx, Ddx19, Ddx21, Dnmt2, Dqx1, Hdac7a, Lce-pending, Mgat1, mouse homologue of CILP, mouse homologue of NOH61, Nudel-pending, Pah, Pdi1, Ppia, Prps1, Ptgds, and Vars2, encoding enzymes; Dagk4, mouse homologue of PSK, Nme2, Snf1lk and Tyki, encoding kinases; Inpp4a and Inpp5b, encoding phosphatases; Il16, Prg, and Scya4, encoding secreted factors; Akap7, Api5, Arfrp1, Arhgap14-pending, Cish2, Dapp1, Fabp6, Fkbp8, Fliz1-pending, Hint, Ier5, Jundp2-pending, Lmo6, Mid1, mouse homologue of AKAP13, mouse homologue of BIN2, mouse homologue of CEZANNE, mouse homologue of CHD2, mouse homologue of MBLL, mouse homologue of SLC16A10, mouse homologue of SLC16A6, mouse homologue of SLC17A5, mouse homologue of TAF5L, mouse homologue of UlSNRNPBP, mouse homologue of ZNF8, Mtap7, Myolc, Nkx2-3, Nsf, Pcdh9, Pkig, Prdx2, Pscd1, Psmb1, Psme1, Psme2, Rg11, Ril-pending, Sax1, Slc14a2, Slc7a1, Slc7a11, Swap70, Txnip, and Ubl3, encoding signaling proteins; Clic3, Gt11-13, mouse homologue of NOL5A, and Vdac2, encoding structural proteins; ABT1-pending, Ctbp1, Dermo1, Ebf, Elf4, Ldb1, mouse homologue of NR1D1, mouse homologue of ZER6, Rest, Tbp, Zfp238, Zfp287, and Zfp319, encoding proteins involved in transcriptional regulation; Lrrc2, Satb1, Slfn4, and genomic regions with the following Celera identification codes mCG10290, mCG10613, mCG11234, mCG11325, mCG11355, mCG11803, mCG11817, mCG12566, mCG12630, mCG12824, mCG13346, mCG14143, mCG14155, mCG14342, mCG15141, mCG15321, mCG16761, mCG16858, mCG17127, mCG17140, mCG17142, mCG17547, mCG17569, mCG17751, mCG17799, mCG17802, mCG17918, mCG18034, mCG1850, mCG18663, mCG18737, mCG20276, mCG20905, mCG20994, mCG21403, mCG21505, mCG21529, mCG21530, mCG21803, mCG22014, mCG22045, mCG22386, mCG2258, mCG22772, mCG23032, mCG23035, mCG23069, mCG23075, mCG23120, mCG2543, mCG2824, mCG2947, mCG3038, mCG3729, mCG3760, mCG50409, mCG50651, mCG5068, mCG5070, mCG51393, mCG52252, mCG52498, mCG53009, mCG53724, mCG55023, mCG55075, mCG55198, mCG55265, mCG55512, mCG56069, mCG56089, mCG56746, mCG57132, mCG57265, mCG57617, mCG57827, mCG58254, mCG58345, mCG5900, mCG5905, mCG59368, mCG59375, mCG59533, mCG59662, mCG59810, mCG59997, mCG60526, mCG60833, mCG61221, mCG61661, mCG61897, mCG61907, mCG61943, mCG62177, mCG62971, mCG63537, mCG63601, mCG64346, mCG64382, mCG64398, mCG65022, mCG65585, mCG65785, mCG66128, mCG66379, mCG66776, mCG66965, mCG7831, mCG7856, mCG8424, mCG9002, mCG9537, mCG9538, mCG9791, mCG9792, mCG9843, mCG9875, mCG9877, and mCG988, or a human homologue thereof.

53. A method of using at least one murine genomic region listed in claim 52 or its human homologue for the preparation of an inhibitor capable of inhibiting the transcription product or activity of a polypeptide encoded by said region or affected by transformations in said region, wherein said inhibitor is chosen from the group consisting of an antibody, an antisense molecule, an RNAi molecule, a ribozyme and a small molecule interfering with the biological activity of the polypeptide encoded by said genomic region or with the biological activity of a polypeptide the expression of which is affected by transformations in said genomic region.

54. An isolated nucleic acid sequence comprising at least one murine genomic region listed in claim 52 or its human homologue, further comprising one or more regulatory sequences.

55. A recombinant vector comprising the nucleic acid sequence of claim 54.

56. A recombinant host cell comprising the vector of claim 55, wherein said host cell is a hematopoietic stem cell.

57. An inhibitor compound capable of inhibiting the transcription product or polypeptide encoded by at least one murine genomic region listed in claim 52 or its human homologue or capable of inhibiting the transcription product or polypeptide the expression of which is affected by a transformation in said genomic regions, wherein said inhibitor compound is an antibody or derivative thereof directed against said polypeptide, and wherein said polypeptide is expressed on the cell membrane.

58. An inhibitor compound according to claim 57, wherein said derivative is selected from the group consisting of scFv fragments, Fab fragments, chimeric antibodies, bifunctional antibodies, intrabodies, other antibody-derived molecules, an antisense molecule, an RNAi molecule, a ribozyme and a small molecule interfering with the biological activity of said polypeptide.

59. An inhibitor compound according to claim 57, for use in the treatment of acute myeloid leukemia (AML).

60. A method of making a pharmaceutical composition for the treatment of cancer, comprising admixing an inhibitor compound according to claim 57 with at least one pharmaceutically acceptable excipient.

61. The method according to claim 60, wherein the inhibitor is incorporated in an amount effective to treat an indication for which gene therapy is indicated.

62. The method according to claim 60, wherein the inhibitor is selected for the treatment of inflammatory diseases.

63. A pharmaceutical composition for the treatment of cancer, comprising at least one inhibitor compound according to claim 57 and a suitable excipient, carrier or diluent.

64. A method of treating a mammal, comprising administering to a mammal the pharmaceutical composition of claim 63 in an amount effective to alleviate or prevent the formation of cancer.

65. A method of treating a mammal, comprising administering to a mammal the hematopoietic stem cell of claim 56 in an amount effective to alleviate or prevent the formation of acute myeloid leukemia.

66. A method of using of at least one murine genomic region listed in claim 52 or its human homologue or a transcription product thereof or polypeptide encoded thereby for the preparation of a diagnostic reagent for diagnosis of cancer.

67. A diagnostic reagent capable of specifically binding to a murine genomic region listed in claim 52 or its human homologue or a transcription product thereof or polypeptide encoded thereby.

68. A diagnostic composition comprising a diagnostic reagent according to claim 67.

69. A method of using a diagnostic composition according to claim 68, for the diagnosis of cancer selected from the group consisting of solid tumor of lung, colon, breast, prostate, ovarian and acute myeloid leukemia (AML).

70. A method of using a diagnostic composition according to claim 69, wherein the diagnosis is performed by means of histological analysis of tissue specimens using specific antibodies directed against encoded polypeptides, using in situ hybridisation analysis of gene expression levels in tissue specimens with RNA probes directed against gene sequences or using polynucleotide or oligonucleotide arrays.

71. A kit comprising diagnostic composition according to claim 68, and one or more reagents selected from the group consisting of reagents for the isolation of nucleic acid fragments from a sample, reagents for the isolation of polypeptides from a sample, reagents for immunostaining of a sample, reagents for in situ hybridisation of a sample and reagents for performing nucleic acid array hybridisations.

72. A method for the development of an inhibitor compound according to 57, comprising the steps of:
a) identification of genes involved in cancer;
b) validation of one or more of the identified genes as potential target gene(s) for the inhibitor compound by one or more of the following methods:
confirmation of the identified gene by Northern Blot analysis in cancer cell-lines;
determination of the expression profile of the identified gene in tumors and normal tissue;
determination of the functional importance of the identified genes for cancer;
c) production of the expression product of the gene; and
d) use of the expression product of the gene for the production or design of an inhibitor compound.

73. A method for the identification of genomic regions involved in the development of cancer comprising the steps of:
a) performing retroviral insertional mutagenesis of a subject, comprising infecting said subject with a tumor inducing retrovirus;
b) isolating chromosomal DNA from tumors developed in the infected subject;
c) digesting said chromosomal DNA with a restriction enzyme capable of cutting at least once in the DNA sequence of said tumor inducing retrovirus and at least once in the chromosomal DNA of said subject;
d) ligating the digested DNA to circular DNA;
e) amplifying the chromosomal DNA fragment flanking the retroviral DNA sequence by performing a first PCR reaction with said circular DNA using a first set of retrovirus-specific primers and performing a second nested PCR with the product of said first PCR reaction using a second nested set of retrovirus-specific primers, and
f) directly determining the nucleotide sequence of said chromosomal DNA fragment, and optionally comparing said nucleotide sequences with known sequences in a database to yield the genomic region involved in the development of cancer.

74. A method for the identification of common virus integration sites in the development of cancer comprising the steps of
a) performing the method of claim 73;
b) designing genomic region-specific amplification primers;
c) isolating nucleic acids from at least two tumors to be analysed for the presence of a common virus integration site;
d) performing an amplification reaction with said nucleic acids using a set of nested primers comprising genomic region-specific primers and retrovirus-specific primers, and
e) blotting the amplification products and separately hybridizing the resulting blot with a retrovirus-specific probe and a genomic region-specific probe to determine the presence of common virus integration sites between said tumors.

75. A set of genomic regions obtainable by a method according to claim 74, wherein said genomic regions comprise at least 2 murine genomic regions selected from the group consisting of Adam11, Akap7, Arpgap14 , Bomb, Cd24a, Cish2, Cig5, Clic3, Cra, Dermo1, EMILIN, Flj20489, Galnt5, Hook, Ier5, IL16, Iprg1, Itgp, Kcnk5, lrrc2, Ltb, Mb11, Mrc1, Mtap7, Ninj2, Nrld1, Pcdh9, Prdx2, Prps1, Pdi1, Ptgd3, Rgl1, Sardh, Scya4, Slc16A6, Swap70, Txnip, Trim46, Tnfrsf17 and Ub13, more preferably comprising at least 2, murine genomic regions selected from the group consisting of Cd24a, Cish2, Cra, Ltb and Prdx2.
